(19) United States
(12) Patent Application Publication
(10) Pub. No.: US 2022/0401031 A1
(43) Pub. Date: Dec. 22, 2022

(54) WIRELESSLY TRIGGERED DEVICE

(71) Applicant: National University of Singapore, Singapore (SG)

(72) Inventors: S Y John HO, Singapore (SG); Viveka KALIDASAN, Singapore (SG); Xin YANG, Singapore (SG); Ze XIONG, Singapore (SG)

(21) Appl. No.: 17/775,614

(22) PCT Filed: Nov. 9, 2020
(86) PCT No.: PCT/SG2020/050643

§ 371 Date: May 10, 2022

(30) Foreign Application Priority Data
Nov. 11, 2019 (SG) 10201910511X

(51) Int. Cl. A61B 5/00; A61B 17/06; A61B 5/07

(52) U.S. Cl. CPC A61B 5/6883; A61B 17/06166; A61B 5/07; A61B 2017/00022

(57) ABSTRACT

A wirelessly triggered device for implantation in vivo is disclosed herein. In a described embodiment, the wirelessly triggered device comprises an electrically conductive suture; and an electronic circuit coated with a biocompatible encapsulating material and communicatively coupled to the electrically conductive suture, the electronic circuit arranged to convert a received wireless triggering signal into an electrical signal for passing through the conductive suture. A reader for use with the device and an electrically conductive surgical thread is also disclosed, among other aspects.

### TECHNICAL FIELD

The present invention relates to wirelessly triggered devices, in particular but not exclusively, devices for in-situ sensing and transmission in remote locations, especially in vivo.

### BACKGROUND

After bodily trauma such as surgery, a cut, infection or other injury, conditions can develop that can, among other things, hinder healing or necessitate further intervention. For example, wound dehiscence can result in the need to replace sutures.

Such conditions can be particularly difficult to detect when they are internal. For internal surgical procedures, dehiscence, infection and other conditions can often only be identified after symptoms indicate the occurrence or presence of the condition. In such cases, the condition may cause patient discomfort or make more drastic procedures necessary.

In addition, even where no adverse condition occurs, it can be very difficult to assess the condition of an internal surgical site to determine, for example, how well the site is healing. Further, it may be desirable to provide additional support to the healing process at the site of interest without further surgical intervention.

It is desirable therefore to provide a means for earlier identification of conditions, to overcome or ameliorate at least one of the above-described problems, to enable surgeons to more confidently identify healing, or at least to provide a useful alternative.

### SUMMARY

In a first aspect, a wirelessly triggered device for implantation in vivo is provided, the device comprising: an electrically conductive suture; an electronic circuit coated with a biocompatible encapsulating material and communicatively coupled to the electrically conductive suture, the electronic circuit arranged to convert a received wireless triggering signal into an electrical signal for passing through the conductive suture.

By wirelessly triggered it is meant that one or more operations of the device is initiated by the arrival of a wireless signal such as an electromagnetic signal, for example a radiofrequency signal, or a magnetic signal, at the device.

The term electrically conductive suture is intended to mean a thread which is capable both of apposing tissue portions and carrying an electrical signal. Preferably, the conductivity of the electrically conductive suture is greater than 100 S/m.

Advantageously, the wirelessly triggered device of the present invention enables the enhancement of a surgical suture via an electrical signal, enabling the provision of sensing capability directly at the site of a wound and/or other functionalities to support wound healing at a site via the suture.

The electrically conductive suture may comprise a first conductive portion; a second conductive portion; and an insulating portion between the first and second conductive portions, the electronic circuit being communicatively coupled to the first and second conductive portions and arranged to pass the electrical signal through the first and second conductive portions. Preferably, the electrically conductive suture comprises an electrically conductive surgical thread comprising an inner surgical thread; an electrically conductive coating on the inner surgical thread, the electrically conductive coating comprising a bio-compatible conductive polymer; and a bio-compatible protective coating on the electrically conductive coating. Such surgical threads provide good electrical conductivity without compromising on strength and pliability.

Alternatively, the electrically conductive suture may comprise a stainless steel surgical thread.

The suture may have a length between 1 mm and 1 m. The suture may have a length between 1 mm and 50 cm. Preferably, the suture has a length of greater than 20 mm. Preferably the suture has a length of less than 30 mm. Preferably, the suture has a length of approximately 25 mm. Sutures of this length enable greatest power transmission.

Preferably the device the device is a passive electronic device. This enables the device to be employed for long periods of time without having to implant an additional power source into the body.

The biocompatible encapsulating material may be selected from PDMS, silicone, parylene-C, and polyurethane.

The electrically conductive suture may be arranged to receive the wireless triggering signal, and the electronic circuit may include a modulating circuit operable to modulate the received wireless triggering signal to produce a backscatter response signal having a specific harmonic as the electrical signal, for transmission by the electrically conductive suture. This arrangement advantageously enables wireless sensing at the site of a wound without implanting an additional antenna. The sensing functionality provided by the device may be further enhanced by the device further comprising a detector operable to detect a predetermined condition at the site, and wherein the modulator is operable to modulate the backscatter signal based on the detected predetermined condition. The detector may include a passive component of the modulating circuit. Advantageously, such sensing devices enable monitoring of an internal or external wound using only a simple low-power or passive device and a suture as an antenna, thereby minimising the number of devices to be implanted into a patient.

The predetermined condition may comprise one or more adverse physiopathological states such as bleeding, infection such as bacterial growth, gastric juice leakage and anastomotic leakage. The modulating circuit may be an RLC circuit and a resonance frequency of the RLC circuit may vary based on the detected predetermined condition, for example, the passive component may be a capacitor or inductor of the RLC circuit.

Preferably, the device further comprises a support member for supporting a layer of responsive material which is susceptible to undergo a change in the predetermined condition, the support member configured to support the responsive material over the passive component. The support member comprises a plurality of relief structures projecting from the modulating circuit and defining a cavity for receiving the responsive material, such as pillars and/or walls or any other type of relief structure capable of supporting a responsive material in place. The presence of a coating of the responsive material over the passive component enables the modulating circuit to respond to one or more physiopathological states in a controlled and predictable manner. Preferably, the responsive material is a hydrogel, such as a DNA hydrogel susceptible to degradation in the presence of nuclease secreted by bacteria, a peptide hydrogel susceptible to degradation in the presence of pepsin, or a heme hydrogel susceptible to solidification in the presence of blood.

Alternatively, or additionally, the electronic circuit of the device may include a rectifier operable to rectify the wireless triggering signal to produce an electrical current as the electrical signal, the electrical current being passed through the conductive suture. In this embodiment, the device may further comprise an antenna for receiving the wireless triggering signal, the rectifier being communicatively coupled to the antenna. The antenna may be the electrically conductive suture or a further electrically conductive suture. Devices according to this embodiment advantageously enable wireless powering at the site of a wound. For example, the device may configured to stimulate a nerve by passing the electrical current through the conductive suture or the suture may be in electrical connection with an electronic device and configured to supply power to the electronic device.

In a second aspect, a wirelessly triggered sensing device for monitoring conditions at a site is provided, the device comprising: a detector operable to detect a predetermined condition at the site; a modulating circuit configured to be communicatively coupled to an antenna, the modulating circuit operable to modulate a wireless triggering signal received at the antenna to produce a backscatter response signal having a specific harmonic, for transmission by the antenna, based on the detected predetermined condition, wherein the detector includes a passive component of the modulating circuit.

Advantageously, such sensing devices provide a simple low-power or passive device for monitoring conditions at a remote location. A component of the modulating circuit itself is employed as the detector, thus requiring no further detecting components to be employed ensuring the size, complexity and cost of the sensing device is minimised. Preferably, the device is a passive electronic device, thereby enabling long term monitoring without requiring an additional power source.

The device may further comprise an antenna and the modulating circuit and/or the antenna may be printed onto a printed circuit board.

The device may be adapted for implantation in vivo, for example at the site of a wound, in which case, the modulating circuit is coated with a biocompatible encapsulating material which may be selected from PDMS, silicone, parylene-C, and polyurethane. In this case, the device may further a connector for connecting the device to a wound closure device in order to enable implantation in vivo, which may be a mechanical connector, such as stitching or stapling, or a chemical adhesive such as glue. The wound closure device may comprise sutures, staples, surgical zips, endoscopic clips, etc. Alternatively, the antenna may be an electrically conductive component of a medical device, which may be one or more of a bandage, a valve, a prosthesis, and an implant.

In cases where the device is adapted for implantation in vivo, the predetermined condition may comprise one or more adverse physiopathological states such as bleeding, infection such as bacterial growth, gastric juice leakage and anastomotic leakage. The modulating circuit is an RLC circuit and a resonance frequency of the RLC circuit may vary based on the detected predetermined condition, for example, the passive component may be a capacitor or inductor of the RLC circuit.

In other embodiments, the predetermined condition may comprise a non-pathological condition such as vital sign measurement, such as heart rate monitoring or pulse monitoring, or suture breakage.

Preferably, the device further comprises a support member for supporting a layer of responsive material which is susceptible to undergo a change in the predetermined condition, the support member configured to support the responsive material over the passive component. The support member comprises a plurality of relief structures projecting from the modulating circuit and defining a cavity for receiving the responsive material, such as pillars and walls. The presence of a coating of the responsive material over the passive component enables the modulating circuit to respond to one or more pysiopathological states in a controlled and predictable manner. Preferably, the responsive material is a hydrogel, such as a DNA hydrogel susceptible to degradation in the presence of nuclease secreted by bacteria, a peptide hydrogel susceptible to degradation in the presence of pepsin, or a heme hydrogel susceptible to solidification in the presence of blood.

In alternative embodiments, the device may be intended for inclusion in food packaging and the predetermined condition may be bacterial growth in food. In this embodiment, the device may further comprise a support member for supporting a layer of responsive material which is susceptible to undergo a change in the predetermined condition, the support member configured to support the responsive material over the passive component. The support member comprises a plurality of relief structures projecting from the modulating circuit and defining a cavity for receiving the responsive material, such as pillars and/or walls or any other type of relief structure capable of supporting a responsive material in place. The presence of a coating of the responsive material over the passive component enables the modulating circuit to respond to one or more conditions in a controlled and predictable manner. Preferably, the responsive material is a hydrogel, such as a hydrogel susceptible to change in the presence of food-borne bacteria.

A reader may be provided for use with a wirelessly triggered device, the wirelessly triggered device comprising: an electrically conductive suture; an electronic circuit coated with a biocompatible encapsulating material and communicatively coupled to the electrically conductive suture, the electronic circuit arranged to convert a received wireless triggering signal into an electrical signal for passing through the conductive suture, the electrically conductive suture being arranged to receive the wireless triggering signal, and the electronic circuit includes a modulating circuit operable to modulate the received wireless triggering signal to produce a backscatter response signal having a specific harmonic as the electrical signal, for transmission by the electrically conductive suture. Alternatively, the reader may be provided for use with a wirelessly triggered device comprising: a detector operable to detect a predetermined condition at the site; a modulating circuit configured to be communicatively coupled to an antenna, the modulating circuit operable to modulate a wireless triggering signal received at the antenna to produce a backscatter response signal having a specific harmonic, for transmission by the antenna, based on the detected predetermined condition, wherein the detector includes a passive component of the modulating circuit. In either case, the reader may comprise: a transmitter configured to transmit a plurality of interrogation signals configured to stimulate a backscatter response signal from the device; a receiver configured to receive the backscatter response signal from the device; and a processor configured to determine a condition at a site based on the backscatter response signal. The reader may be provided together with a wirelessly triggered device as a sensing platform.

In a third aspect, an electrically conductive surgical thread for use as a suture to appose tissue portions is provided, the surgical thread comprising: an inner surgical thread; an electrically conductive coating on the inner surgical thread, the electrically conductive coating comprising a bio-compatible conductive polymer; and a bio-compatible protective coating on the coating.

Advantageously, surgical threads according to embodiments enable electrical conductivity to be incorporated into a surgical suture, without compromising on the flexibility or strength of the suture.

The bio-compatible conductive polymer may comprise one or more of PEDOT:PSS, poly(pyrrole); polythiophene; poly(3-alkylthiophene); polyphenylene-vinylene; polyaniline; and poly(p-phenylene sulfide). Preferably, the bio-compatible conductive polymer comprises PEDOT:PSS which provides good conductivity while maintain excellent pliability. The bio-compatible conductive polymer comprises three or more layers of PEDOT:PSS in order to optimise conductivity.

The bio-compatible protective coating may comprise Parylene-c and the inner surgical thread may comprise silk. The inner surgical thread may comprise any material. However, surgical threads according to embodiments having an inner silk thread have been shown to provide sutures with particularly good signal to noise ratio when employed as antennas. The surgical thread may have any thickness. However, surgical threads according to embodiments having a diameter of U.S.P. size 0 have been shown to provide sutures with particularly good signal to noise ratio when employed as antennas.

In a fourth aspect, a method of producing an electrically conductive surgical thread is provided, the electrically conductive surgical thread comprising an inner surgical thread; an electrically conductive coating on the inner surgical thread, the electrically conductive coating comprising a bio-compatible conductive polymer; and a bio-compatible protective coating on the coating is provided, the method comprising: providing a surgical thread; coating the thread with an electrically conductive coating comprising a bio-compatible conductive polymer; and encapsulating the coated thread in a biocompatible material. Advantageously, this method provides a surgical thread with high conductivity. Preferably, applying an oxygen plasma treatment to the provided thread before removing wax from the thread. Preferably, the electrically conductive coating is vacuum dried.

The inner surgical thread may be a medical-grade suture thread. In the alternative, the inner surgical thread may be a commercially available thread.

In a fifth aspect, a method of monitoring conditions at a site in vivo is provided, the method comprising: implanting a device into the site, the device comprising an electrically conductive suture; an electronic circuit coated with a biocompatible encapsulating material and communicatively coupled to the electrically conductive suture, the electronic circuit arranged to convert a received wireless triggering signal into an electrical signal for passing through the conductive suture, the electrically conductive suture being arranged to receive the wireless triggering signal, and the electronic circuit includes a modulating circuit operable to modulate the received wireless triggering signal to produce a backscatter response signal having a specific harmonic as the electrical signal, for transmission by the electrically conductive suture; transmitting a plurality of interrogation signals configured to stimulate a backscatter response signal from the device; receiving the backscatter response signal from the device; and determining a condition at the site based on the backscatter response signal. This method advantageously provides an accurate non-invasive method of monitoring conditions in vivo.

The condition may comprise one or more physiopathological conditions. The one or more physiopathological conditions includes one or more of healing, bleeding, infection, dehiscence, suture breakage, heart rate and respiration rate. Implanting the device comprises may comprise suturing at least a portion of a wound with the electrically conductive surgical suture.

In an embodiment, a method of monitoring conditions at a site in vivo is provided, the method comprising: implanting a device into the site, the device comprising a detector operable to detect a predetermined condition at the site; a modulating circuit configured to be communicatively coupled to an antenna, the modulating circuit operable to modulate a wireless triggering signal received at the antenna to produce a backscatter response signal having a specific harmonic, for transmission by the antenna, based on the detected predetermined condition, wherein the detector includes a passive component of the modulating circuit. The condition may comprise one or more physiopathological conditions. The one or more physiopathological conditions includes one or more of healing, bleeding, infection, dehiscence, suture breakage, heart rate and respiration rate. Implanting the device may comprise connecting the device to a medical device at the site.

In a fifth aspect, a transmission assembly is provided, the transmission assembly comprising: a transmission device comprising: an antenna connector for connecting to an antenna, and a signal generator for generating a signal for transmission using the antenna when the transmission device is attached thereto, wherein the signal generator has an unaffected condition and an affected condition, and predetermined condition around the transmission device cause the signal generator to transition to the affected condition, the signal when generated by the signal generator in the unaffected condition being different to the signal if generated by the signal generator when in the affected condition; and an antenna connected to the signal generator by the antenna connector, wherein the antenna comprises a conductive suture.

Advantageously, certain embodiments of the invention use an electrically conductive suture as an antenna for transmitting a signal. This enables the transmission device or transmission assembly to leverage off a suture that is already necessary to insert into a patient.

Since the devices according to embodiments can be tailored to detect a specific condition—e.g. blood leakage/bleeding/haemorrhage, bacterial infection, wound dehiscence, etc.—for transitioning the generator device to the affected condition, the devices are capable of indicating a specific condition within a patient, using the suture as an antenna, or outside the patient where, for example, the antenna forms part of or is sewn into a wound dressing.

Harmonic backscattering embodiments can eliminate the use of wires, except to the extent that a conductive suture or similar, being already necessary to provide even in the absence of the device, can be considered a wire.

Devices according to embodiments may be transponders or radio frequency identified tags for long-term, battery monitoring. Such devices can be readily made biocompatible and are cheap and easy to use in view of the present teachings.

### BRIEF DESCRIPTION OF THE DRAWINGS

Embodiments of the present invention will now be described, by way of non-limiting examples, with reference to the drawings in which:

FIG. 1 shows a system for monitoring conditions in a remote location according to an embodiment;

FIG. 2 shows a first example of a sensing device according to an embodiment comprising an RLC circuit in series;

FIG. 3 shows a second example of a sensing device according to an embodiment comprising an RLC circuit in parallel;

FIG. 4 shows a third example of a sensing device according to an embodiment comprising an integrated antenna;

FIG. 5 shows an example of a modulator according to an embodiment attached to a surgical suture;

FIG. 6 shows a first example of a modulator for attaching to a surgical suture according to an embodiment;

FIG. 7 shows a second example of a modulator for attaching to a surgical suture according to an embodiment;

FIG. 8 shows a schematic representation of a sensing device according to an embodiment employed in vivo;

FIG. 9 shows a method of monitoring a wound site using a sensing device according to an embodiment;

FIG. 10 shows a method of measuring vital signs using a sensing device according to an embodiment;

FIG. 11 shows the device of FIG. 7, with antenna threaded through the device and a hydrogel coated on the capacitor;

FIG. 12 shows a method for producing a DNA hydrogel according to an embodiment;

FIG. 13 shows the degradation of the DNA hydrogel of FIG. 12 in the presence of nuclease;

FIG. 14 shows a method for producing a peptide hydrogel according to an embodiment;

FIG. 15 shows the degradation of the peptide hydrogel of FIG. 14 in the presence of pepsin;

FIG. 16 shows a process for detecting gastric leakage using the peptide hydrogel of FIG. 14;

FIG. 17 shows a schematic of a reader for use with a sensing device according to an embodiment;

FIG. 18 shows a schematic of a reader for use with a sensing device according to an embodiment;

FIGS. 19(a) and 19(b) show an antenna for use with the reader of FIG. 18;

FIG. 20 shows a system for rectifying power at a remote location in accordance with an embodiment;

FIG. 21 shows a first example of a rectifying device according to an embodiment;

FIG. 22 shows a method of fabricating an electrically conductive surgical thread according to an embodiment;

FIG. 23 shows the receiving power of sutures according to embodiments as a function of the length of the suture for three stitch patterns;

FIG. 24 shows the power received as a function of conductivity and diameter of sutures according to embodiments;

FIG. 25 shows illustrates the transfer efficiency of sutures according to embodiments as a function of frequency, and resonant frequency changes for different capacitor capacities;

FIG. 26 shows stress as a function of strain for a number of commercially available sutures compared with a suture produced in accordance with an embodiment;

FIG. 27 shows the Tissue Drag Force for a number of commercially available sutures compared with a suture produced in accordance with an embodiment;

FIG. 28 shows the change in resistance of a WISE suture produced in accordance with an embodiment as the suture was subjected to mechanical cycles of contraction and elongation;

FIG. 29 shows the change in resistance of sutures produced in accordance with an embodiment measured over three weeks in physiological buffer 1X phosphate buffer solution (PBS);

FIG. 30 shows the cell viability for sutures produced in accordance with embodiments;

FIGS. 31(a) and 31 (b) show the change in capacitance resulting from exposure of DNA hydrogel layered on a capacitor to the extracellular nuclease secreted by Staphylococcus aureus bacteria compared with a control;

FIG. 32(a) shows the normalised power as a function of frequency for three different severities of bleeding using a WISE suture produced according to embodiments in combination with a modulator;

FIG. 32(b) shows the power as a function of frequency using a WISE suture produced according to embodiments in combination with a modulator for different types of suture breakage;

FIG. 33(a) shows the inflammation scores over time for wounds closed with sutures according to embodiments;

FIG. 33(b) shows the healing scores over time for wounds closed with sutures according to embodiments;

FIG. 34 shows the change in resonance frequency over time for wounds closed with sutures to which are fixed modulators according to embodiments;

FIG. 35 shows the resistance of sutures prepared in accordance with the protocols of Table 1;

FIG. 36 shows the effect of the number of coatings of PEDOT:PSS on the resistance of sutures according to embodiments;

FIG. 37 shows images of sutures produces according to embodiments with different sizes;

FIG. 38 shows images of sutures produces according to embodiments with different base sutures;

FIG. 39 shows signal and noise measurements for the sutures of FIG. 37;

FIG. 40 shows signal and noise measurements for the sutures of FIG. 38;

FIG. 41 shows the wireless reflection coefficient S11 as a function of frequency for the antenna shown in FIGS. 19(a) and (b);

FIGS. 42(a), 42(b) and 42(c) show simulated harmonic spectra for Lembert, Lock-stitch and Cushing stiches using surgical thread according to embodiments, respectively;

FIG. 43 shows the simulated capacitance of a modulating circuit according to an embodiment loaded with a cylinder shape of peptide hydrogel;

FIG. 44 shows the simulated capacitance of a modulating circuit in accordance with an embodiment in contact with cylindrical shape media of different types;

FIG. 45 shows the effect of the addition of nuclease on the resonant frequency of a simulated modulating circuit coated with a layer of DNA hydrogel;

FIG. 46 shows the effect of the addition of 10 μL DI water on the resonant frequency of a simulated modulating circuit coated with a layer of DNA hydrogel;

FIGS. 47(a), 47(b) and 47(c) demonstrate dynamic vital sign monitoring using a simulated WISE suture according to an embodiment before and after skin closure, gastric solution injection and suture breakage, respectively;

FIGS. 48(a), 48(b) and 48(c) show the frequency spectra obtained from WISE sutures according to embodiment before and after skin closure, exposure to gastric solution, and suture breakage, respectively;

FIG. 49 shows a comparison of the amplitude of a backscatter signal measurement taken using a WISE suture according to an embodiment compared with an ECG signal; and

FIG. 50 shows the signal to noise variation over the 14 days for skin and muscle.

### DETAILED DESCRIPTION

For purposes of brevity and clarity, descriptions of embodiments of the present disclosure are directed to a sensor device, conductive suture and reader, in accordance with the drawings. While aspects of the present disclosure will be described in conjunction with the embodiments provided herein, it will be understood that they are not intended to limit the present disclosure to these embodiments. On the contrary, the present disclosure is intended to cover alternatives, modifications and equivalents to the embodiments described herein, which are included within the scope of the present disclosure as defined by the appended claims. Furthermore, in the following detailed description, specific details are set forth in order to provide a thorough understanding of the present disclosure. However, it will be recognized by an individual having ordinary skill in the art, i.e. a skilled person, that the present disclosure may be practiced without specific details, and/or with multiple details arising from combinations of aspects of particular embodiments. In a number of instances, well-known systems, methods, procedures, and components have not been described in detail so as to not unnecessarily obscure aspects of the embodiments of the present disclosure.

In embodiments of the present disclosure, depiction of a given element or consideration or use of a particular element number in a particular figure or a reference thereto in corresponding descriptive material can encompass the same, an equivalent, or an analogous element or element number identified in another figure or descriptive material associated therewith.

References to “an embodiment/example”, “another embodiment/example”, “some embodiments/examples”, “some other embodiments/examples”, and so on, indicate that the embodiment(s)/example(s) so described may include a particular feature, structure, characteristic, property, element, or limitation, but that not every embodiment/example necessarily includes that particular feature, structure, characteristic, property, element or limitation. Furthermore, repeated use of the phrase “in an embodiment/example” or “in another embodiment/example” does not necessarily refer to the same embodiment/example.

The terms “comprising”, “including”, “having”, and the like do not exclude the presence of other features/elements/steps than those listed in an embodiment. Recitation of certain features/elements/steps in mutually different embodiments does not indicate that a combination of these features/elements/steps cannot be used in an embodiment.

As used herein, the terms “a” and “an” are defined as one or more than one. The recitation of a particular numerical value or value range herein is understood to include or be a recitation of an approximate numerical value or value range.

The ability to non-invasively monitor and communicate the turn of events happening in a remote area such as a surgical site will pave way to the development of next generation smart sensing technologies. Disclosed herein is a wireless sensing (WISE) technology for wirelessly transmitting information about a remote site (e.g. a surgical site) to an external reader. In some embodiments, this is achieved through harmonic backscattering, which can eliminate wires through the integration of a highly miniaturized transmission device such as a transponder. In some embodiments, the technology is alternatively employed to provide wireless powering of processes designed to support healing at a site.

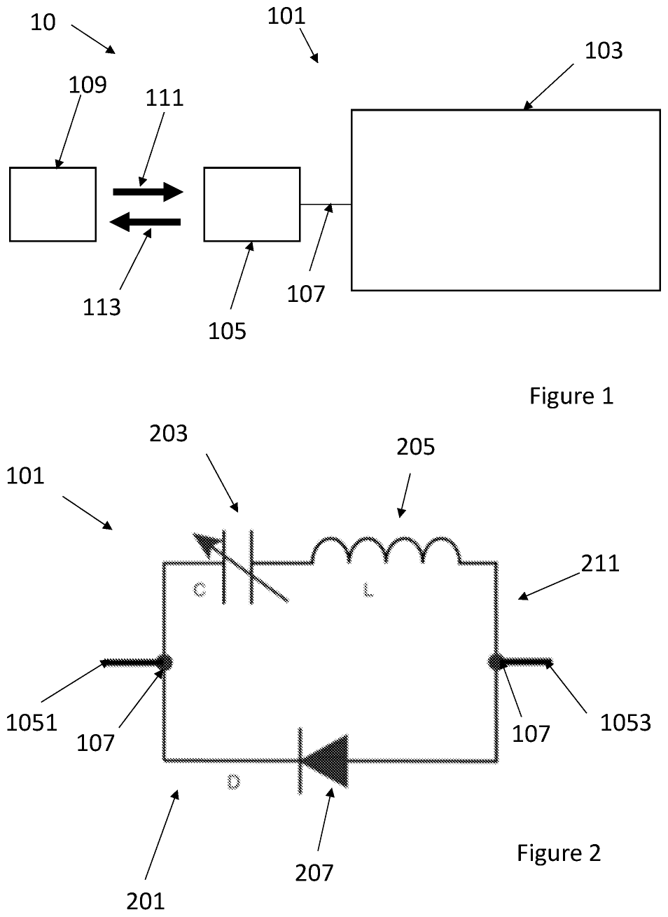

FIG. 1 shows a schematic representation of a system 10 for sensing events occurring in a remote area or site according to an embodiment. The system 10 comprises a sensing device 101 including an antenna 105 and a modulator 103 in communicative connection 107 with the antenna 105. The system 10 further comprises a reader 109 which may be remote from and communicatively coupled to the device 101. The antenna 105 may be configured to receive a signal 111 emitted by the reader 109, equivalently an interrogation signal. As will be clear from the embodiments discussed below, the signal may be a radiofrequency signal, a magnetic field or any other signal of known or identifiable characteristics, such as frequency and amplitude. The signal 111 may further be capable of providing power to the modulator 103. This process will be discussed further below in association with particular embodiments. The interrogation signal may stimulate the antenna to transmit a response signal 113. In a particular embodiment, the response signal may be the result of backscatter coupling between the antenna 105 and the reader 109, i.e. the response signal 113 may be a backscatter signal. In another embodiment, the response signal 113 may be stimulated by inductive coupling between the reader 109 and antenna 105, e.g. when the reader and antenna both comprise induction coils.

The connection 107 between the antenna and the modulator may simply comprise an electrical contact—e.g. metal to metal contact—or may comprise a weld or solder or any other form of electrical connection.

In an embodiment, the power of the device 101 may be provided by a battery or energy harvester device. In a particular embodiment, the device 101 is passive, i.e. it comprises no power source, nor does it comprise any physical connection (e.g. wires) to a power source. In this embodiment, power to the device is instead provided wirelessly, for example via the received signals 111 (either by inductive or backscatter coupling) received or via other wireless charging methods.

In an embodiment, the modulator 103 is configured to modulate, i.e. to alter, at least one characteristic of the backscatter signal 113. In an embodiment, it may be configured to alter the frequency spectrum of the signal. Thus, in an embodiment, the sensing device 101 may be configured to receive a signal 111 of given characteristics, alter one or more characteristics of the signal and backscatter it with the altered characteristics as response signal 113. In an embodiment, further alteration of the signal characteristics may occur in addition to modulation by the modulator 103, at the antenna 105.

In an embodiment, the modulation, or alteration of the characteristics of the response signal applied by the modulator 103 (and antenna 105, where appropriate) are dependent on the conditions in which the device 101 resides. For example, the modulator 103 may be sensitive to or otherwise affected by liquids, gasses, or other substances in contact with it, sensing device 101, or a particular portion of sensing device 101. Likewise, the antenna 105 may be sensitive to or otherwise affected by conditions in which it resides. In some embodiments, the device may not output a signal at all according to the conditions. The sensing device 101 or a portion of the sensing device 101 may be specially adapted to alter its behaviour when a particular event occurs.

Thus, the sensing device 101 may be employed to monitor for the occurrence of at least one event, the event being associated with a change in the conditions surrounding the device. The reader 109 may receive the response signal 113 and, from the characteristics of the response signal, determine that the event has occurred or is occurring.

The sensing device 101 therefore could be said to have an unaffected condition and an affected condition, the unaffected condition is a condition indicating that the conditions sought to be monitored by the sensing device 101 are not yet present. In the affected condition, the event sought to be monitored has occurred, or is occurring. In the affected condition, the device 101 either no longer transmits a signal or transmits a different signal to that which it would transmit in the unaffected condition. This means predetermined conditions (i.e. the occurrence of the event) around the device 101 cause the sensing device 101 to transition to the affected condition such that the signal output by the device 101 in the unaffected condition is different to the signal output by the device 101 when in the affected condition. Thus, the absence of a signal or a changed signal is indicative of the event having taken place.

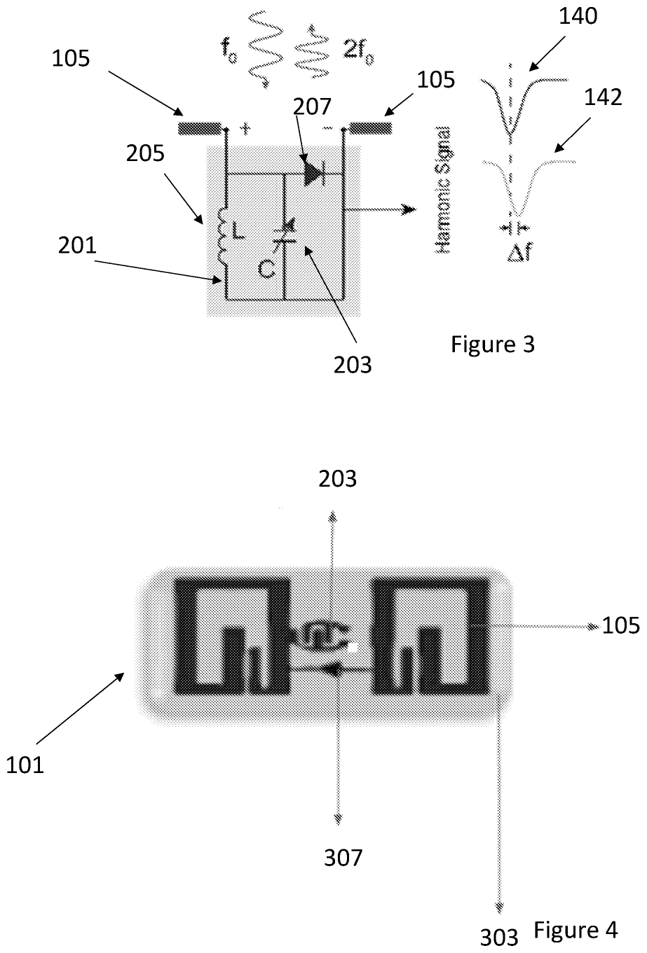

FIG. 2 shows a circuit diagram of device 101 according to an embodiment. In this embodiment, the modulator 103 comprises a modulating circuit 201, which, in this embodiment is an RLC circuit comprising a capacitor 203, an inductor coil 205 and a diode 207 connected in series. In other embodiments (see, for example FIG. 3) they may be connected in parallel.

In the embodiment of FIG. 2, the antenna 105 is connected across the modulating circuit 201 for receiving and/or transmitting signals. It should be appreciated that the antenna comprises two separate conductive parts 1051 and 1053, which are connected via the modulating circuit 201, in order for the device to function as described. When the antenna 105 receives a signal oscillating at the resonance frequency f₀ of the circuit 201, the modulating circuit 201 will cause the antenna 105 to backscatter the signal at the second harmonic of the resonance frequency 2f₀, i.e. the signal is modulated as the change in second harmonic resonant frequency. The resonance frequency of the circuit 201 is dependent on the inductance of the inductor 205, the capacitance of the capacitor 203 and the resistance or impedance in the circuit.

Thus, a typical sensing measurement by the reader 109 employed with device 101 may be performed by sweeping the signal from a first frequency to a second frequency, for example 1 to 2 GHz, with identical amplitudes and simultaneously recording the 2nd harmonic by a spectral analyser. It will be appreciated that the frequency range of the signal may vary but preferably includes the resonance frequency of the RLC circuit of the device in its unaffected condition.

In an embodiment, one or more of the circuit parameters of modulating circuit 201 is configured to alter in response to the conditions in which the circuit 201 resides. In an embodiment, the capacitor 203 is configured such that its capacitance alters in response to the conditions in which the device 101 resides. For example, the capacitance of the capacitor 203 may alter due to contact of a substance with the device 101 or with the portion of the device comprising the capacitor 203. A change in the capacitance alters the resonance frequency of the circuit and therefore the frequency spectrum of a signal 113 backscattered by the antenna 105 will shift. This is shown schematically in FIG. 3 where the resonance frequency (the minimum in the spectrum) of spectrum 140 is shifted to a higher frequency in 142.

Likewise, a change in the capacitance of the modulating circuit 201 would cause a change in the magnetic field induced by inductor 205, in the case of inductive coupling with the reader 109.

In other embodiments, the sensing device 101 may be configured such that other components, such as the inductor 205, alter their electrical properties in the presence of one or more external substances. Similarly, the sensing device 101 may be configured such that the resistance of the circuit is affected by a particular event.

The exact values and form of the circuit 201 may change in a manner that will be clear in view of the present teachings, while maintaining the functionality described herein. In particular, a change in circuit parameters or characteristics—e.g. resistance or impedance, capacitance, inductance and others—that is either a step change or progressive, will indicate a transition to the affected condition. Notably, the affected condition may constitute a range of signals for, for example, progressive conditions such as bacterial infection, or may be a fixed change such as the removal of a single in the event of antenna breakage.

In embodiments, the power of the sensing device 101 may be provided by a small battery, energy harvester device, or, in particular, may be generated by current flowing through the antenna 105 on capture of signal 111 or by another passive charging mechanism such as an electromagnetic field (EMF) applied by the reader 109 or other remote device concurrently with or comprising signal 111. In other embodiments, the reader 109 produces an EMF that charges the sensing device to cause it—i.e. the modulator thereof—to emit a signal. The modulator 103 may instead be activated by a remote device, the remote device activating the modulating circuit 201 comprised within the modulator 103 by transmitting a signal of a resonant wavelength of the modulating circuit 201, that is captured by the antenna 105. That captured signal may stimulate a response in the device, being either battery driven or passively charged as mentioned above.

In some embodiments, the sensing device 101 is a highly miniaturised transponder for transmitting information from a remote site, such as deep tissue, to an external wireless reader. The transponder may use harmonic backscattering. The transponder may instead send a signal of a different frequency or amplitude, or signal of mixed frequencies, that is not an harmonic of the stimulus signal—i.e. that supplied by the remote device or remote system to stimulate a response from, or activate, the transponder.

In other embodiments, the sensing device 101 is an RFID tag charged by an applied electromagnetic field to emit a signal. The present technology can therefore also be used to activate the tag/transponder on-demand to perform desired monitoring activities in and around the site of sensing device 101.

The sensing device 101 according to embodiments enables efficient and secure transmission of wireless signals between the sensing device (e.g. transponder), which could be an implant, and an external reader to provide information about the remote area being monitored by the sensing device. In the embodiment shown, changes in the environment around the sensing device are sensed by a change in resistance, capacitance or inductance (RLC) of modulating circuitry, the properties of the circuit therefore change as the modulator transitions to the affected condition. This results in modulation of the signal transmitted by the sensing device 101 and may manifest in a change in resonant frequency. The sensing device therefore enables non-invasive monitoring of remote sites such as surgical sites within the body to provide real-time continuous or on-demand information.

FIG. 3 shows another example of sensing device 101 comprising an RLC based modulating circuit 201 as the modulator 103 in which the inductor and capacitor are arranged in parallel. Otherwise, the functioning of the device is analogous to that of FIG. 2.

FIG. 4 shows the design of a sensing device 101 according to an embodiment in which all components, both antenna 105 and modulator 103 (as well as connection 107) are incorporated in the same component as a flexible printed circuit board 303. The sensing device 101 comprises an interdigital capacitor 203, an inductor-Schottky diode 307 and a printed antenna 105. In an embodiment, the printed circuit board 303 may be encapsulated or coated with a material permitting its use in a particular environment, for example, a biocompatible silicone polymer to enable use in the human or animal body. In embodiments, the thickness or type of encapsulating material or coating may be chosen to enable materials in contact with the encapsulating material to alter the permittivity of the capacitor 203 or the electrical properties of other components, thereby enabling an electrical response of the circuit to conditions external to the device. The exact thickness will vary according to the application and the particular material employed. However, in general, the thinner the encapsulating material, the greater the responsiveness to the electrical properties of the circuit to any bodily fluid such as blood, gastric fluid, wound pus, etc. Preferably, the thickness of the encapsulating material is in the range 1 micrometers-5 cm. Preferably, it is greater than 400 micrometers in thickness. Preferably it is less than 600 micrometers in thickness. Preferably the thickness of the encapsulating material is approximately 500 micrometers.

In embodiments the printed circuit board may be further coated or partially coated with a material which is responsive to a particular substance. This will be discussed further below.

In embodiments, the antenna 105 may not be integrated into the same component as the modulator 103 and instead comprise a separate component to which the modulator 103 is connected, or not included at all, as discussed above. Such embodiments will be discussed in detail below.

In a particular embodiment, the sensing device 101 or a portion of the sensing device 101 (such as, for example, the modulator 103) is configured for attachment to a wound closure device including, but not limited to, surgical staples, sutures, bandages, surgical gauze, zips, endoscopic clips, etc. The device may be mechanically (e.g. via stitching or stapling) or chemically (e.g. via glue) attached to the wound closure device. The sensing device 101 is compatible with any medical implant, including but not limited to orthopaedic, breast and cardiac, etc. implants to monitor the site of the implant and also with devices such as catheters, drain bags, etc., to monitor entry and exit sites for complications.

In particular embodiments, the sensing device 101 as a whole, or the just the modulator 103, are configured for attachment to a surgical suture. The device may be attached to the suture by threading the suture through fixtures in the deice. Alternatively, or additionally, the device could also be attached to the suture by clamping or with a suitable adhesive.

In such embodiments, the sensing device 101 may be configured to monitor for an event related to the wound and/or surgical suture. For example, the sensing device 101 itself may monitor for the occurrence of at least one event, such as bleeding, dehiscence, infection, leakage or, in more positive aspects, healing.

The sensing device 101 (or modulator 103, as appropriate), may be attached after placement of the suture for example by clamping to the suture or with an adhesive, for example surgical glue or strips. In other embodiments, the sensing device 101 or modulator 103, as appropriate, may be incorporated into the suture or attached in advance of placement of the suture, for example by stitching or threading the suture through fixtures on the device. Moreover, more than one device 101 or modulator 103 may be attached to a single suture, such that each sensing device (for the case of plural devices) or each modulator (for the case of multiple modulators for one sensing device) monitors one of many different conditions.

Devices according to embodiments may be produced using conventional techniques for producing printed circuit boards (PCBs), such as chemical etching of copper foil laminated to an insulating substrate with one or more components mounted in electrical connection with the copper on the surface of the PCB. In an embodiment, the capacitor is a printed interdigital capacitor.

In an embodiment, the sensing device 101, or a portion of the sensing device, such as the modulator 103 and/or antenna 105 may be encapsulated by biocompatible material such as a biocompatible silicone polymer in order to prevent unwanted side-effects inside the human or animal body. In an embodiment, the PCB is coated with the encapsulation material to the desired thickness. Preferably, the biocompatible material is selected from PDMS, silicone, parylene-C, and polyurethane.

For attachment to a wound closure device, such as a suture, the size of the device is preferably in the range 0.1 mm to 20 cm and the weight of the device is preferably in the range 1 g to 20 g.

In an embodiment, the sensing device 101, or modulator 103, may be attached to a conventional surgical thread (e.g. one that is commercially available) or to a specially adapted surgical thread. In an embodiment, the modulator 103 is attached a surgical thread which is, or a portion of which is electrically conductive. In this embodiment, the surgical thread itself may act as the antenna or a portion of the antenna 105 (such as the component 1051 or 1053 only on one side of the circuit) for the sensing device 101.

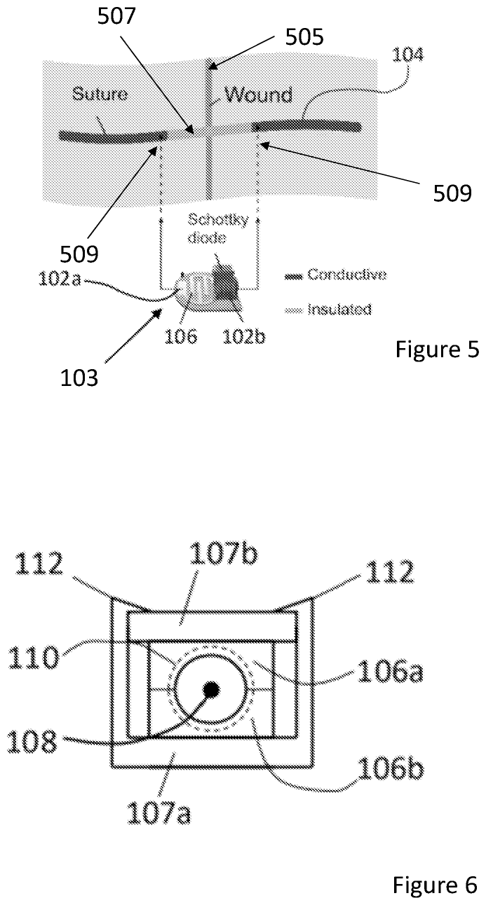

An arrangement according to this embodiment is shown in FIG. 5 in which a modulator 103 according to an embodiment comprising RLC circuit 106 is connected to a partially electrically conductive suture 104 arranged across a wound 505 in order to hold it closed. The modulator 103 comprises antenna connectors 102a and 102b for connecting to the suture 104 at connection points 509 fixed by an adhesive or by mechanical clamping. In this embodiment, the suture forms both portions of the antenna 1051 and 1053 and consequently has an insulating portion 507 between the connection points 509, being conductive outside of this portion. Preferably, the conductivity of the conductive portions of the electrically conductive suture are above 100 S/m to ensure that the suture is capable of functioning well as an antenna.

Thus, in this embodiment, the suture 104 itself acts as the antenna 105 (e.g. a dipole antenna) for receiving a signal 111 and backscattering a modulated signal 113, and the circuit 106 of the modulator 103 modulates the signal for transmission using the suture when the transmission device is attached to it, in accordance with embodiments described above.

Thus, in this embodiment, the modulator 103 and suture 104 together comprise the sensing device 101 according to an embodiment.

Advantageously, employing the modulator 103 in a surgical context using the suture 104 as an antenna results in minimal additional devices being implanted during the surgery since the suture is already required—i.e. use of an additional antenna is avoided. In addition, compromise of the suture, such as breakage can be monitored via the signal modulated by the modulator 103. This will be discussed in detail below.

In an embodiment, the antenna connectors 102a and 102b may comprise one or more solder pads for solder attachment to the suture/antenna 104. As shown in FIG. 6, the antenna connector 102 may instead include one or more metal blades 107a, 107b that together form an annulus 108. In the case of a surgical suture antenna, the blades 107a, 107b may penetrate a protective cover (the location of the protective cover post-penetration being indicated by broken line 110) of the antenna to contact the conductive suture thereunder. The blades are mounted to jaws 107a, 107b, jaw 107a having clips 112 that are received around the other jaw 107b to hold the two together.

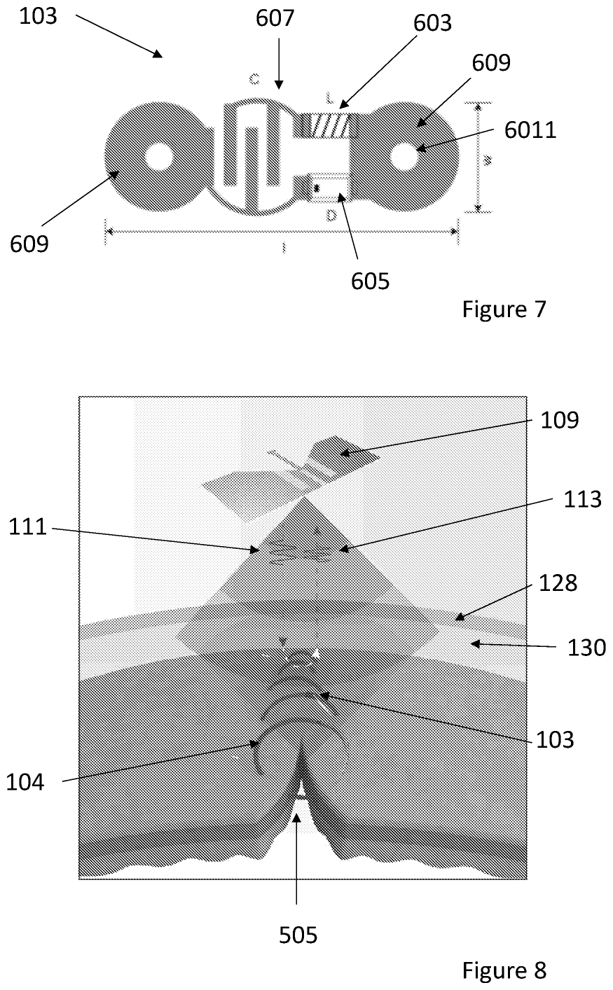

FIG. 7 shows a schematic layout of a modulator 103 according to another embodiment configured for attachment to a conductive suture. The layout is suitable for printing on a flexible printed circuit board comprising the components.

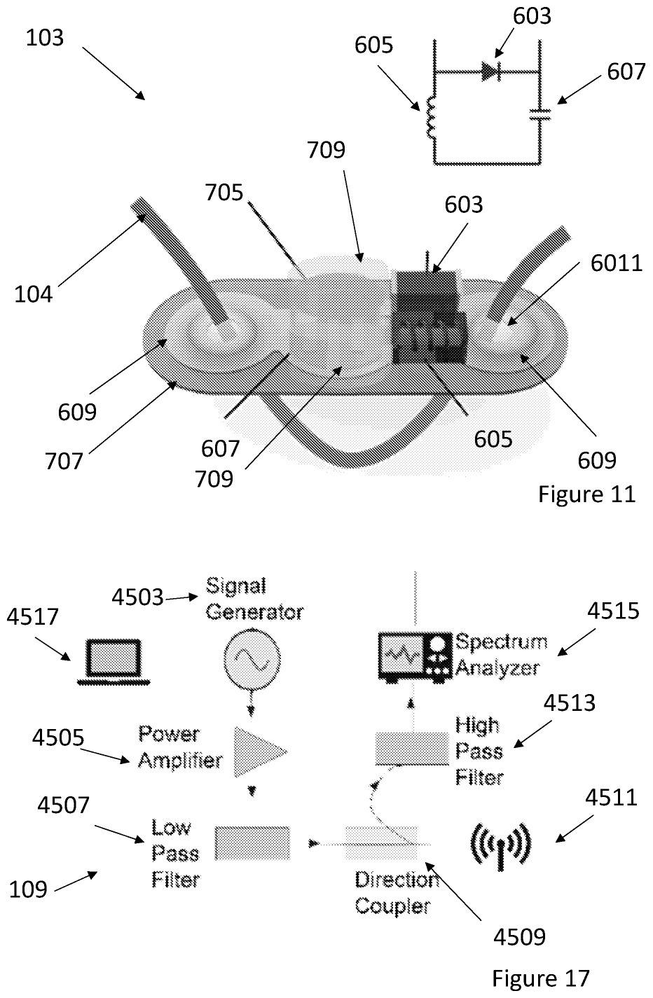

The modulator 103 comprises a circuit having an interdigital capacitor portion 607, inductor 603 and diode 605. The modulator 103 further comprises hollow electrodes 609 for contact with a conductive suture 104 which may be encapsulated by medical grade silicone, according to requirements. The suture 104 is threaded through the holes 6011 in the electrodes. This is shown schematically in FIG. 11, which shows an alternative view of the device of FIG. 7. The suture is encapsulated by a biocompatible material such as parylene-C and the electrodes are secured to the suture by adhesive or mechanical clamping. In this embodiment the electrodes are pre-patterned. As before, the suture comprises an insulating portion (not shown) between the two conductive portions threaded through holes 6011 in order to enable its function as both portions of the antenna of the device.

Suitable components for use in the embodiment of FIG. 7 or other embodiments described here are commercially available, such as from Wurth Elektronik (e.g. 12 nH inductor 74765112A) and SKYWORKS SOLUTIONS (Schottky diode SMS7630-079LF). The dimensions of the modulator of FIG. 7 may be as small as 6 mm (l)×2 mm (w).

FIG. 8 shows a schematic representation of a sensing device in accordance with embodiments of the present invention being employed to monitor for events involving or around a sutured wound.

In this embodiment, a modulator 103 comprising a modulating circuit 201 according to an embodiment is mounted to a suture 104. In an embodiment, a reader 109 generates a signal 111 at frequency f₀. The signal 111 penetrates the skin 128 and tissue 130 of a patient and is captured by the suture 104 which acts as an antenna while also closing surgical wound 505. The length of the suture 104 may be specifically designed to receive a signal of a particular frequency f₀ or a range of frequencies. A modulator 103 attached to the suture, generally near the mid-point of the suture 104, receives the signal and generates a response signal 113—i.e. a signal from the modulator 103—the frequency of which is an harmonic of signal 111—e.g. 2f₀. The reader 109 captures signal 113 and determines from the frequency (or other parameters of the signal the creation or modulation of which may be used as desired—e.g. phase shift indicated between the phase of the original signal 111 and response signal 113 from the antenna, as shown in FIG. 3) whether the event has occurred—i.e. the modulator 103 has transitioned to the affected state.

Events that modulator 103 may be adapted to monitor for include (but are not limited to) bleeding involving blood leakage or other liquid saturation of the transmission device, leakage of gastric juices, compromise of the antenna (such as breaking of the suture), or bacterial infection or bacterial growth, anastomotic leakage and healing.

In an embodiment, the modulator 103 itself may or may not be specifically adapted to monitor for a particular event, according to requirements. For example, in the case of bacterial infection, the modulator 103 may have a coating that is susceptible to consumption by bacteria—such as a bacterium-specific deoxyribonucleic acid (DNA) hydrogel. When no bacterial infection is occurring, the coating will remain intact. Consequently, circuitry of the modulator 103 (particularly the modulating circuit 201) will remain unaffected. When a bacterial infection occurs, circuitry of the modulator 103 will become progressively more exposed to the bacteria or surrounding tissue, or both. The circuitry therefore becomes affected resulting in a change in output of the modulator 103. This change may be a change in frequency of the signal resulting from, for example, a change in capacitance or inductance of the modulating circuit 201 comprised within the modulator 103. This change may be progressive such that, in the event of use of a coating for bacterial consumption, the signal gradually changes as more of the coating is consumed and the circuitry is increasingly affected.

In the case of antenna compromise—e.g. breakage of the suture 104 in the presence of dehiscence—the modulator 103 may simply be unable to send a signal and therefore, in this and other cases, the change in signal may be that no signal is transmitted. In other embodiments, similar coatings are provided that are susceptible to consumption, degradation, removal or change by other, non-bacterial agents. Exemplary devices according to these embodiments will be discussed below.

The bleeding, infection and compromise in suture integrity discussed above can be post-surgical complications that are able to be monitored by a sensing device according to embodiments.

According to embodiments described above the conductive suture can be used to appose incisional wounds on the skin and deep inside the body with devices attached either before or after suturing.

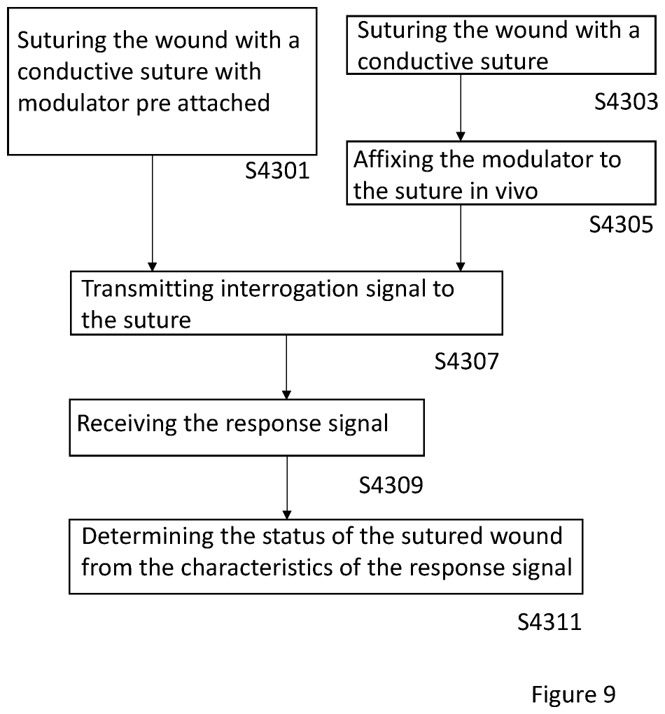

FIG. 9 shows a flowchart for a general method of monitoring a surgical wound according to an embodiment. In step S4301 a conductive suture 104 according to an embodiment to which a modulator 103 according to an embodiment is fixed is employed to suture the wound, or a part of the wound. Alternatively, in step S4303, a conductive suture 104 is employed to suture the wound or a portion of the wound and then, in step S4305, the modulator 103 is attached to the suture in vivo. Subsequently, in step S4307 a reader transmits an interrogation signal to the suture. The interrogation signal may include the resonance frequency of a modulating circuit 201 comprised in the modulator 103 in one or both of the unaffected condition and the affected condition. The wavelength of the interrogation signal may be capable of inducing backscattering from the suture 104.

In step S4309, the response signal (e.g. the backscatter signal) is received from the suture and the characteristics of the signal are analysed, for example, the frequency spectrum of the signal may be analysed. In step S4311 determination is made as to whether a particular event, including but not limited to bleeding, dehiscence, suture breakage, bacterial infection, gastric leakage or anastomotic leakage is occurring or has occurred at the wound site.

Healing of the wound site may be determined by an absence in the change in the signal, indicating the lack of any complication.

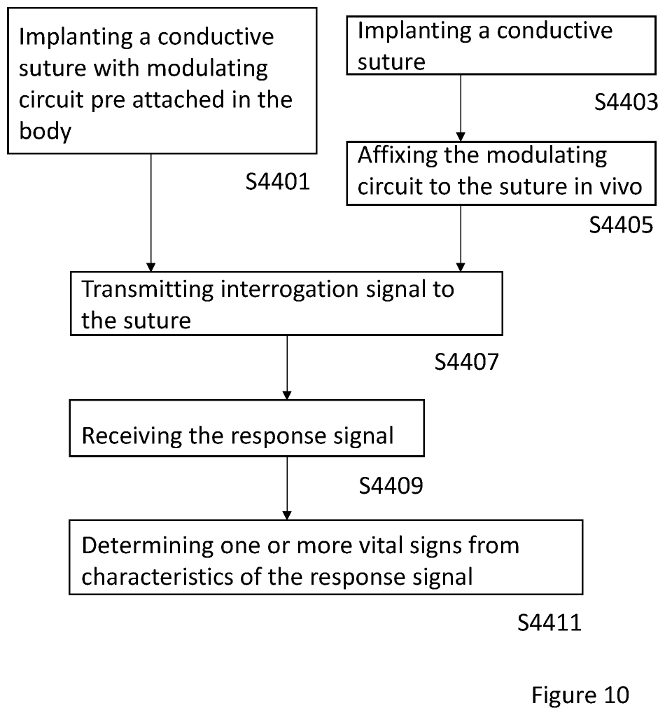

In an embodiment, a modulator and suture may be employed instead of, in addition to, or even after (e.g. after coating that is susceptible to consumption by bacteria has been fully consumed) determining the occurrence of a particular event at a wound site, to monitor the vital signs of a patient. This is possible because physiological processes such as breathing and heartrate will alter the distance between the antenna (the suture) and the reader. This may be particularly useful for monitoring of patients following surgical procedures involving incisions. Alternatively, a conductive suture 104 could be employed to attach the modulator to the patient body for vital sign monitoring without necessarily being employed to close a wound. A method of measuring vital signs according an embodiment is shown in FIG. 10 and described below.

In step S4401 a conductive suture according to an embodiment to which a modulator 103 is fixed is implanted into the body. Alternatively, in step S4403, a conductive suture is implanted into the body and then, in step S4405, the modulator 103 is attached to the suture in vivo. Subsequently, in step S4407 a reader transmits an interrogation signal to the suture. The interrogation signal may comprise sweeping the signal from a first frequency to a second frequency. The interrogation signal may include the resonance frequency of a modulating circuit 201 comprised within the modulator 103 in one or both of the unaffected condition and the affected condition. The wavelength of the interrogation signal may be capable of inducing backscattering from the suture 104.

In step S4409, the response signal (e.g. the backscatter signal) is received from the suture 104 and the characteristics of the signal are analysed, for example, the frequency spectrum of the signal may be analysed. In step S4411 the amplitude of the signal, which is indicative of the distance of the suture from the reader, are analysed to determine one or more vital signs of the patient.

Thus, in some embodiments, the modulator may be connected to an electrically conductive suture configured to act as an antenna for the modulator. In addition to ensuring that the overall size of the device remains as small as possible as no additional components are required for an antenna, employing a suture as an antenna also enables monitoring for breakage of the suture as, in the case of breakage of the suture the signal produced by the antenna will necessarily change, e.g. by a change in amplitude or the suture being unable to transmit any signal at all. The transmission by the suture may also be affected by bleeding at the site of the wound.

In an embodiment, a conductive suture is employed with a modulator comprising an RLC circuit with no particular adaptation for monitoring for events occurring within the body, i.e. the modulator and suture together are configured only to monitor for disruption of transmission by the suture or the vital signs of the patient. The change in the signal transmitted will therefore only be indicative of such events. In other embodiments, the modulator may be adapted to monitor for a particular event occurring within the body, for example, gastric leakage, as described above. In this embodiment, the device is thus configured to both monitor for breakage of the suture (e.g. in dehiscence) via the suture itself and for other events occurring within the body, via the change in electrical properties of a modulating circuit comprised within the modulator.

As discussed above, in some embodiments, a layer of responsive material is applied to a sensing device according to an embodiment in order to vary the electrical parameters of the modulator 103, for example the modulating circuit 201 comprised within the modulator 103 as the material is degraded, or otherwise altered, in response to conditions surrounding the sensing device. In an embodiment, the layer of material is arranged over the capacitor of the modulating circuit 201.

FIG. 11 shows a schematic representation of the device 103 of the embodiment of FIG. 7, over the top of the capacitor of which is arranged a layer of responsive material 705. The circuit diagram corresponding to the arrangement of this embodiment is also shown in the inset for reference. In this embodiment, the layer of responsive material 705 is arranged in a cylinder shape to match the shape of the capacitor 607.

The responsive material 705 is held in place by two pillars 709 mounted on the surface of the substrate and arranged to hold the responsive material over the capacitor 607, i.e. to provide the necessary mechanical support (as required by the viscosity of the material) to the responsive material in the environment in which the device will be employed, for example in vivo. Preferably, the pillars are formed from Polydimethylsiloxane (PDMS) due to its biocompatibility, however other biocompatible materials could also be used. The pillars are fabricated by 3D printed or using a laser carved template. They may be mounted to the substrate 707 before encapsulation.

It will be appreciated that other relief structures, such as walls could be employed instead of the pillars.

In the embodiment of FIG. 11, the responsive material is arranged above the capacitor 607, in particular over the space between the digits of the capacitor. This arrangement ensures that degradation or other change of the responsive material will result in changes in the permittivity of the capacitor, thereby altering the capacitance of the circuit and altering the resonance of the RLC circuit of which the capacitor 607 is a component. The capacitance variation and sensitivity with a given responsive material may be tuned by changing the gap between the interdigital electrodes and the thickness of the encapsulating material. Preferably the gap between the interdigital electrodes of the capacitor is less than 500 nm for an encapsulation of less than 1 mm.

In an embodiment, the surface of the substrate along with the electrodes may be encapsulated by biocompatible material, such as medical grade silicone, and the responsive material may be arranged on the surface of the encapsulating material.

Although, in FIG. 11 the responsive material is shown as being arranged over the capacitor 607 according to this preferred embodiment, the responsive material could instead, or additionally be arranged over different components of the RLC circuit, for example the inductor 605, where changes to the responsive material will result in changes to the inductance of the inductor 605.

In an embodiment, the responsive material 705 may be a hydrogel, for example a peptide hydrogel which degrades in the presence of peptide, a DNA hydrogel which degrades in the presence of nuclease secreted by bacteria, or a heme hydrogel that solidifies in the presence of blood.

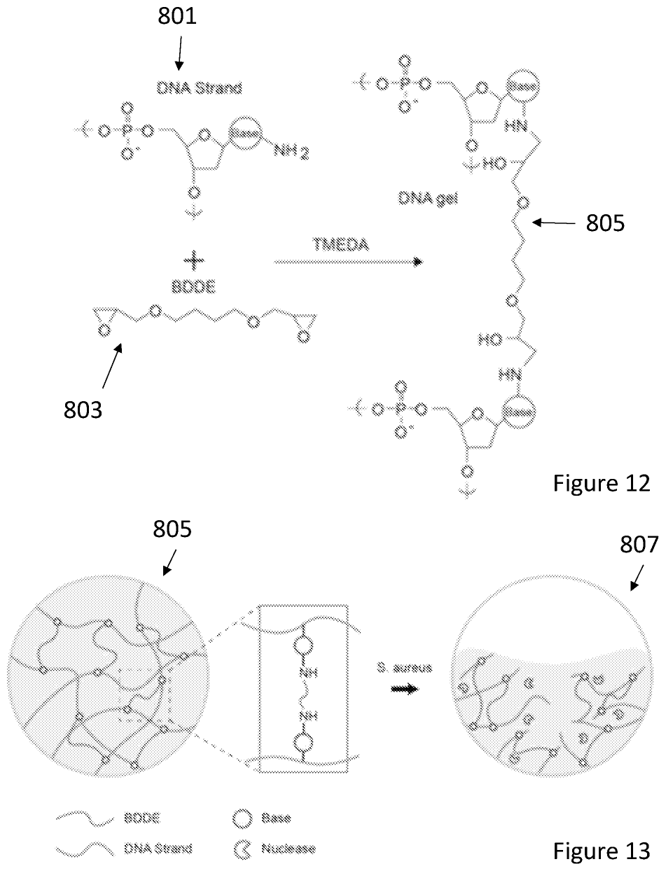

FIG. 12 shows a method of preparing a DNA hydrogel suitable for use with a sensing device according to an embodiment, for example, the sensing device of FIG. 11.

The method comprises mixing a DNA gel precursor 801 with 1,4-Butanediol diglycidylether (BDDE) 803. The presence of N,N,N′,N′-Tetramethylethylenediamine (TMEDA) initiates the amine-epoxy addition and cross-link the DNA strand with BDDE, forming a DNA hydrogel 805.

Preferably, the DNA precursor may be prepared by dissolving 10 wt % deoxyribonucleic acid sodium salt (smDNA) in 4.0 mM NaBr solution and uniformly mixing 2.5 wt % crosslinker, 1,4-Butanediol diglycidyl ether (BDDE), with the precursor and 0.5 wt % N,N,N′,N′-Tetramethylethylenediamine (TMEDA) as the catalyst. This ensures that get appropriate gelation properties and viscosity are obtained for the hydrogel to be held in place with PDMS pillars according to embodiments.

The skilled person will appreciate that other DNA hydrogels could be produced according to other methods according to embodiments.

The DNA hydrogel of this embodiment, is susceptible to digestion by nuclease. Nuclease is secreted by pathogenic bacteria and helps them escape from neutrophil extracellular traps (NETs). NETs are primarily composed of DNA from neutrophils and the secreted nuclease cleaves the backbone of the DNA strand.

Thus, in the presence of bacteria, the cleaving of the DNA strand in the DNA hydrogel causes the collapse of the DNA gel. When the DNA gel is arranged above the capacitor, therefore, the dissipation of the DNA gel due to this mechanism will result in a change in the capacitance of the capacitor, and will therefore be detectable via changes in the electronic properties (specifically capacitance and therefore resonance) of the device according to embodiments, as described above. This is shown schematically in FIG. 13, showing the DNA hydrogel before 805 and after (collapsed state) 807 introduction of nuclease.

Thus, the hydrogel of this embodiment is suitable for use with a device for detecting the occurrence of bacterial infection of a wound site.

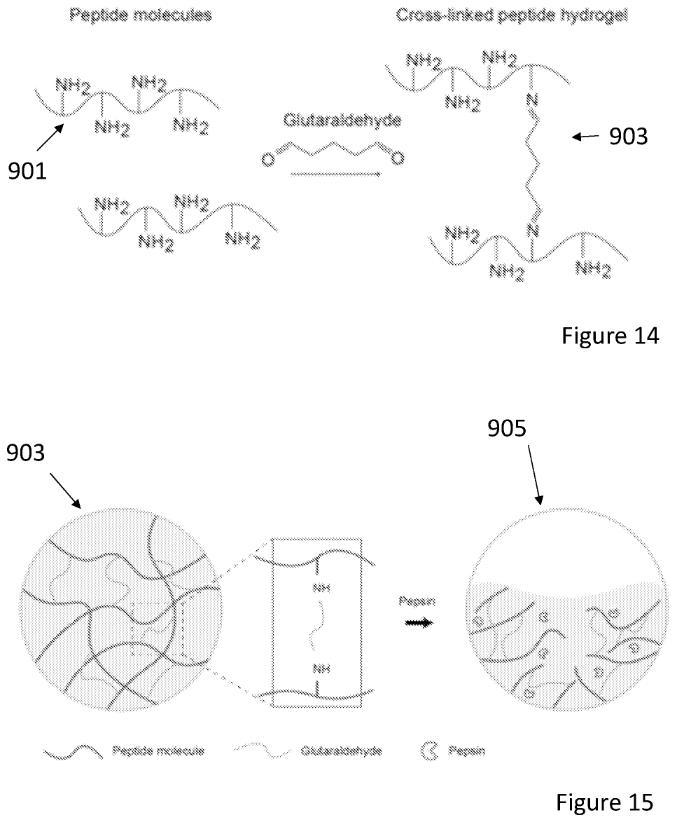

FIG. 14 shows a method of preparing a peptide hydrogel suitable for use with a device according to an embodiment, for example, a sensing device 101 comprising a modulating circuit 201 of which FIG. 11 shows a portion. Preferably, a peptide precursor 901 with 0.5-1 wt. % glutaraldehyde in DI with 1:1 in volume, resulting in a cross-linking reaction, forming a peptide hydrogel 903. This ensures that get appropriate gelation properties and viscosity are obtained for the hydrogel to be held in place with PDMS pillars according to embodiments.

The cross-linking gives the resulting hydrogel 903 a jelly-like appearance which advantageously provides mechanical strength for retaining its structure after coating onto a device according to an embodiment, for example, as shown in FIG. 11.

The skilled person will appreciate that other peptide hydrogels could be produced according to other methods according to embodiments.

The peptide hydrogel of this embodiment is susceptible to digestion by pepsin. Pepsin may be present for example, following gastric leakage, for example following gastric surgery or similar. When the hydrogel is exposed to pepsin, the crosslinked peptide is broken into amino acid components, resulting in collapse of the hydrogel. As discussed above in relation to the DNA hydrogel, the change in hydrogel state may be detected by a device according to an embodiment due to a change in environmental dielectric permittivity of the capacitor. This is shown schematically in FIG. 15 showing the peptide hydrogel before 903 and after 905 (collapsed state) introduction of nuclease.

FIG. 16 shows a schematic of a method of detection of a gastric leak using a modulator 103 comprising a modulating circuit 201 loaded with a peptide hydrogel 903. In step S1001, anastomic leakage occurs from a sutured wound resulting in the leakage of gastric juice. The peptide in the gastric juice results in a reaction of the bioresponsive material (peptide hydrogel) coated on the modulating circuit in accordance with an embodiment. This results in a change in the capacitance of the capacitor in step S1003 and a resulting shift of resonant frequency (Δfᵣ) in step S1005 of the modulating circuit that can be recorded wirelessly by a reader, as described above in accordance with embodiments.

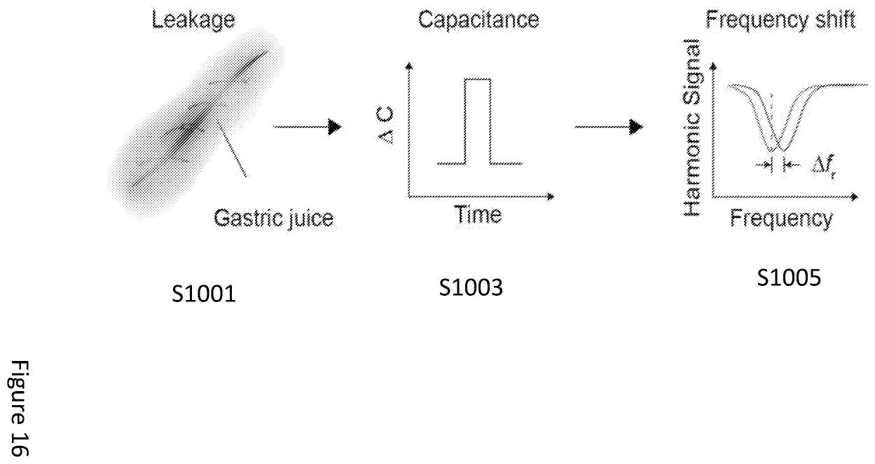

FIG. 17 shows a schematic representation of a reader 109 according to an embodiment suitable for providing an interrogation signal 111 to a sensing device 101 according to embodiments and receiving a response signal 113. The reader comprises a signal generator 4503 which generates a signal which may include a resonant frequency of the modulator (either in the affected or unaffected condition, or both) for which the reader will be employed. Typically, the reader scans frequencies over a range, for example, by sweeping the signal from a first frequency to a second frequency, for example 1 to 2 GHz. The signal is passed through a power amplifier 4505, followed by a low pass filter 4507 and directed by a direction coupler 4509 to the antenna 4511 for transmission. Signals received by the antenna 4511 are directed by the direction coupler 4509 to a high pass filter 4513 and then to a spectrum analyzer 4515 for analysis of the signal enabling determination of the conditions around the device. The output of the spectrum analyser may be interpreted by a user, or the reader 109 may itself further comprise processing circuitry configured to determine if the signal produced by the spectrum analyser is indicative of a condition at the sensing device 101. For example, a processing module of the reader (not shown) may be configured to compare the amplitude of the signal with a threshold value stored in memory. If the amplitude of the signal is below the threshold, the system may determine that there is a breakage in the suture and/or dehiscence and provide an output indicative of this condition, for example, by displaying a warning on a monitor of the system or by outputting an audio signal. The reader may also be configured to record and display the variation in amplitude of the signal with time, for example for vital sign measurement such as heart rate and respiration rate measurement.

In another example, the processor may be configured to compare the frequency spectrum of the received signal with an expected frequency spectrum based on the 2^{nd} harmonic frequency spectrum of the output signal stored in memory. If the frequency spectrum is shifted beyond a threshold value, the system may determine that a condition, such as gastric leakage, anastomotic leakage, bleeding or bacterial infection has occurred, according to the configuration of the device employed.

In an embodiment, the system may be configured to output the characteristics of the signal determined by the spectrum analyzer 4515 to an external device, such as user device 4517 for processing in order to determine of a condition at the site, as described above.

It will be appreciated that determination of a condition at the site of interest from the received backscatter signal could be performed in a number of ways in addition to those described above.

One or more of the components shown in FIG. 17 may be omitted or other components added, in accordance with embodiments.

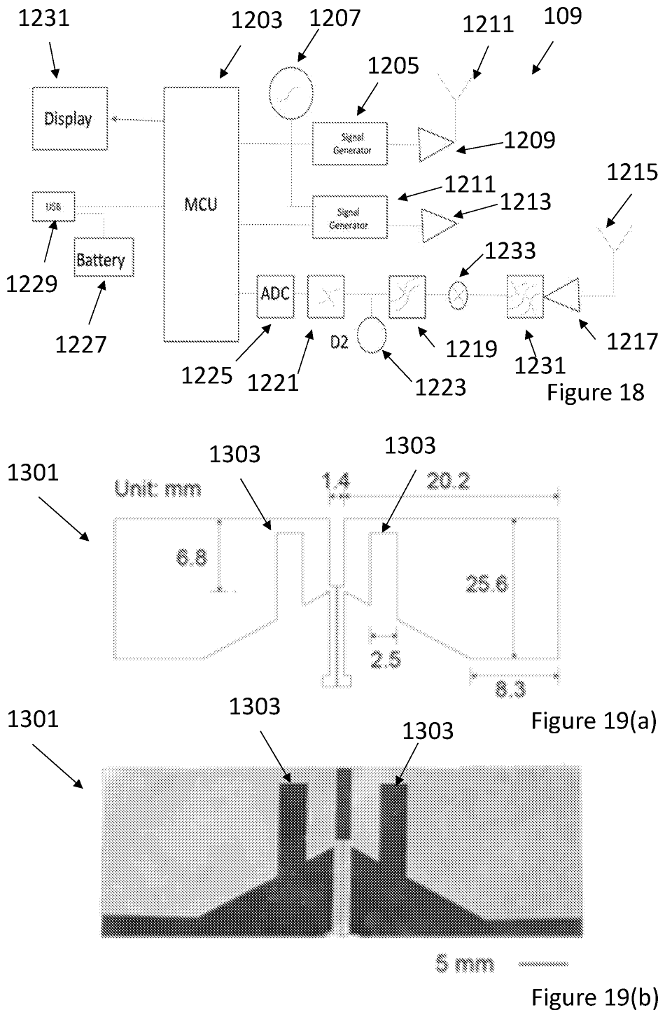

FIG. 18 shows a schematic diagram of reader 109 according to another embodiment for providing an interrogation signal 111 to a sensing device 101 according to embodiments and receiving a response signal 113. In an embodiment the reader 109 is a handheld device for ease of application.

The reader comprises a processor 1203 configured to process data relating to signals received from sensing devices 101 according to embodiments and display data on the display 1231. The device may comprise a battery 1227 and/or USB port 1229 for supplying power to the device. The device comprises a first signal generator 1205 configured to generate radiofrequency signals at the unaffected resonance frequency of the modulating circuit when the antenna (e.g. suture) is intact as indicated by 1207. The signal generator 1205 is connected to amplifier 1209 and antenna 1211 for transmitting the signal produced by signal generator 1211.

The reader further comprises a second signal generator 1211 which acts as a reference for receiving power and also for boosting frequency and/or power.

The reader comprises an antenna 1215 for receiving signals from the sensing device according to embodiments connected to amplifier 1217. The reader may comprise several modules for detecting signals specific to the occurrence of certain events. For example, in the embodiment of FIG. 18, the reader comprises filter modules 1231, 1219 and 1221 for filtering signal characteristics indicative the breakage of the suture stitches either side of the modulator, on one side of the modulator only, or on the stitch on which the modulator is attached, respectively. The filtered signals are subsequently processed by the processor 1203 in order to determine the condition which has occurred.

The reader further comprises an analogue to digital converter 1225, a varactor diode 1223 and a mixer 1233.

FIGS. 19(a) and 19(b) show a schematic and photo, respectively, of an antenna 1301 for use with the reader 109 of FIG. 18 in accordance with a particular embodiment. The design comprises a beveled center-fed planar dipole antenna with slots 1303 added to improve the performance on lower frequency.

The person skilled in the art will appreciate that other antennas could be employed according to embodiments.

In an embodiment, a reader according to the embodiment of FIG. 18 is employed with the sensing device 101 according to the embodiment of FIG. 4 as a platform for monitoring a surgical site. The passive sensing device 101 according to the embodiment of FIG. 4 can be attached to any wound closure device during surgery, at the site of surgery to sense the physiology around the surgical site. The hand-held reader 109 according to the embodiment of FIG. 18 is then employed to power the passive sensing device on-demand and to communicate the events occurring at the surgical site.

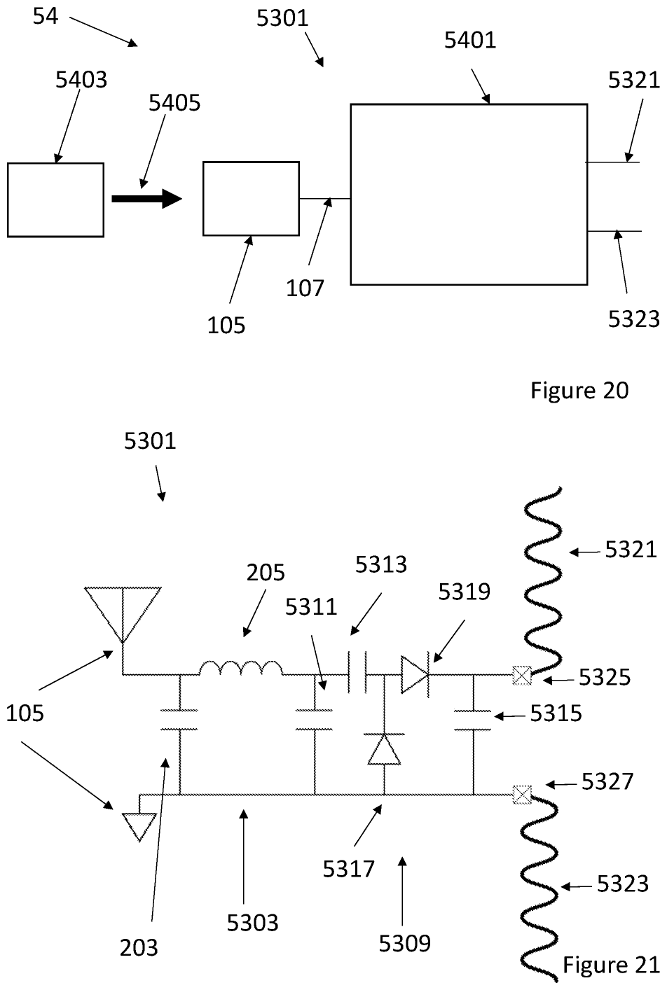

FIG. 20 shows a schematic representation of a system 54 according to another embodiment for providing wireless power to a pair of electrodes 5321, 5323 for providing functionality at a remote site. In an embodiment, the site is in vivo.

The system 54 comprises a wirelessly triggered rectifying device 5301 including an antenna 105 and a rectifying module 5401 in communicative connection 107 with the antenna 105. The system 54 further comprises an emitter 5403 which may be remote from and communicatively coupled to the device 54. The antenna 105 may be configured to receive a triggering signal 5405 emitted by the emitter 5403. As will be clear from the embodiments discussed below, the signal may be a radiofrequency signal, a magnetic field or any other signal suitable for triggering the device. The triggering signal 5405 may further be capable of providing power to the device 5301. The triggering 5405 may cause a potential difference across the electrodes 5321 and 5323 and therefore a current to flow between them when placed in electrical connection.

The connection 107 between the antenna and the rectifier 5401 may simply comprise an electrical contact—e.g. metal to metal contact—or may comprise a weld or solder or any other form of electrical connection.

In an embodiment, the power of the device 5301 may be provided by a battery or energy harvester device. In a particular embodiment, the device 5301 is passive, i.e. it comprises no power source, nor does it comprise any physical connection (e.g. wires) to a power source. In this embodiment, power to the device is instead provided wirelessly, for example via the received signals 5405 or via other wireless charging methods.

FIG. 21 shows the circuit diagram of wirelessly triggered device 5301 according to an embodiment. The device comprises an antenna 105 connected to a Pi-matched circuit 5303 comprising a capacitor 203 and an inductor 205. The Pi-matched circuit itself is connected to a voltage multiplying circuit 5309 configured to rectify signals received by the antenna and the Pi-matched circuit. The Pi-matched circuit and voltage multiplying circuits together make up the rectifying module 5401. In this example, the voltage multiplying circuit comprises capacitors 5311, 5313, 5315 and two diodes 5317, 5319. It will be appreciated that other designs of rectifying circuits could be employed. Electrodes 5321 and 5323 are connected to the rectifying circuit either side of the capacitor 5315 via connectors 5325 and 5327, respectively. In this embodiment, at least one of the electrodes 5321, 5323, and preferably both of the electrodes, is formed form an electrically conductive suture.

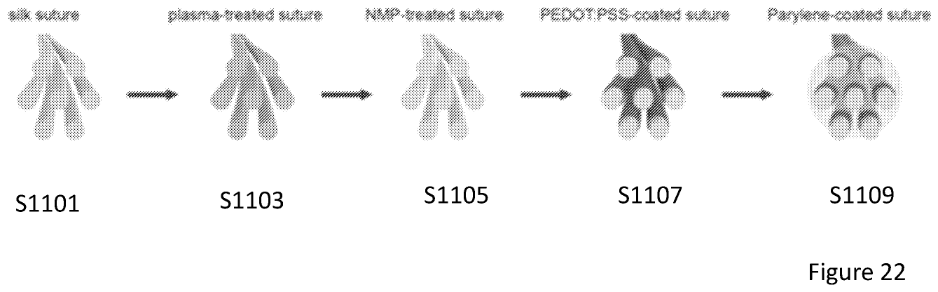

As described above in relation to FIG. 5, in a preferred embodiment, the suture comprises two conductive portions separated by an insulating portion, thereby enabling its use as both electrodes in the embodiment of FIGS. 21 and 22.

As in the case of the embodiments described above, the device 5301 may be produced using conventional techniques for producing printed circuit boards (PCBs), such as chemical etching of copper foil laminated to an insulating substrate with one or more components mounted in electrical connection with the copper on the surface of the PCB or by employing printed components, as appropriate.

In an embodiment, device 5301, or a portion of the device, may be encapsulated by biocompatible material such as a biocompatible silicone polymer in order to prevent unwanted side-effects inside the human or animal body. In an embodiment, the device comprises a PCB coated with the encapsulation material to the desired thickness. Preferably, the biocompatible material is selected from PDMS, silicone, parylene-C, and polyurethane. The antenna 105 may comprise a separate component or components or may be integrated into the same component as that forming the RLC 5303 and/or voltage multiplying circuit 5309, for example, both circuits and the antenna may be printed onto a PCB.

The connections 5325 and 5323 between the electrodes and the rectifying circuit may simply comprise an electrical contact—e.g. metal to metal contact—or may comprise a weld or solder or any other form of electrical connection. The mechanical connection to an electrically conductive suture may be achieved by threading the suture through fixtures in the device, as described above in association with the embodiment of FIG. 11. Alternatively, or additionally, the device could also be attached to the suture by clamping or with a suitable adhesive.

Wireless power received by the antenna 105 of the device 5301 will be modulated by the Pi-matched circuit 5305 which functions as an impedance matching circuit. This power is applied to match the voltage multiplier 5309 which rectifies the received modulated radiofrequency signal causing a potential different between electrodes 5321 and 5323. When placed in vivo, therefore, a current will flow between the electrodes due to the inherent electrical conductivity of human or animal tissue

Thus, in this embodiment, in contrast to those described above, an electrically conductive suture is employed as an electrode.

In an embodiment, an electrically conductive suture is employed both as one or more of the electrodes and as the antenna. This may be achieved by employing two sutures (one forming the antenna and one forming the electrode) or by dividing a single suture into a number of conductive portions, each separated by an insulating portion, in an analogous fashion to the two conductive portions described above in relation to FIG. 5.

By placing the electrodes at a spaced distance in the body and triggering the flow of electrical current though them via a wireless triggering pulse, therefore, the flow of current through the electrodes could be employed in nerve stimulation, by delivering a nerve stimulation pulse. For example, the electrodes may be positioned in the body in order to cause stimulation of the sciatic nerve when a current flows between them. The electrical impulses sent by the suture acting as an electrode can cause a reduction in pain signals being sent to the central nervous system, and therefore the pain experienced by a patient. They may also stimulate the production of endorphins which are natural painkillers produced by the body.

It will be appreciated that the wireless power level and corresponding current for nerve stimulation will depend on the depth at which the device is employed. However, in an example they are at least 1 W and 1 microAmp respectively, which equates to a Specific Absorption Rate (SAR value) of 4 W/Kg.

Thus, the device 5301, enables wireless triggering, via a radiofrequency pulse, of a nerve stimulation pulse.

In other embodiments, the electrodes, 5321 and 5323 or equivalently portions of an electrically conductive suture may be employed as leads in order to power a further device.

In an example, the electrodes are employed as leads for powering an LED and photodetector to be employed as optical sensors in the body, for example for detecting bleeding. Changes in the transmission quotient between the LED and photodetector are indicative of the presence of blood between the two and therefore bleeding, for example, from a sutured wound. The embodiment of FIGS. 20 and 21 may therefore form part of a sensing device.

In another example, the electrodes are employed in drug elution. In this example, the drug may be arranged in a reservoir implanted into the body. The electrodes may then be employed to power a heat generating device, which stimulates the elution of the drug from the reservoir using the change in temperature resulting from the device. Alternatively, the electrodes may be employed to power a light emitting device, such as an LED for stimulating elution of the drug via light. The electrodes could also be employed to electrically stimulate elution of the drug directly from the reservoir.

Thus, in these examples, the device 5301 may be employed as a wirelessly triggered device for stimulating drug elution.

It will be appreciated that the electrodes could be employed as leads for a wide variety of devices providing useful functionality with the body.

Thus, advantageously, devices according to the embodiment of FIGS. 20 and 21 enable wireless powering to monitor, power, stimulate and record the events in remote sites, e.g. nerve repair, stimulation and recording. Thus, devices according to this embodiment provide additional support to the healing process directly at the site of interest without further surgical intervention and with minimal additional devices being implanted during the original surgery since a suture may already be required.

As described above, according to embodiments, electrically conductive sutures may be configured to act as an antennas (e.g. monopole, dipole, helical etc) or electrodes depending on the application.

In an embodiment, the electrically conductive surgical suture comprises a suture formed from a conductive material, such as stainless steel. In a particular embodiment, the electrically conductive suture comprises a surgical suture—e.g. a commercially or otherwise available suture for medical purposes—for apposing tissue portions, which is then coated in a conductive coating. The particular suture which is employed as the inner suture (to which the coating is applied) is not particularly limited. However, suitable examples include sutures made from silk, cotton or vicryl. Examples of specific commercially available sutures suitable for use include prolene and PDSII sutures, and all other commercially available sutures.

The conductive coating ensures signals that an electrical signal can be carried by the suture, thereby enabling its use as an antenna and/or electrode. Preferably, the conductive coating is selected to ensure that the conductivity of the suture is greater than 100 S/m.

The coated surgical suture may then be encapsulated in a protective coating—this may be provided over the length of the suture, or over only that portion to which the modulator is attached. The protective coating may be inert or otherwise non-toxic or non-reactive to surrounding tissue. For example, the protective coating may be biocompatible polymer such as parylene-c. Similarly, the conductive coating can be formed from a non-toxic and non-reactive material, such as a biocompatible conductive polymer. Preferably, the biocompatible conductive polymer is poly(3,4-ethylenedioxythiophene)-poly(styrenesulfonate) (PEDOT:PSS). Advantageously, PEDOT:PSS is a conductive ink that can be adsorbed into the suture material without compromising on the pliability while achieving the desired conductivity.

Other substances to replace the above biocompatible conductive polymer according to embodiments include other conductive polymers such as poly(pyrrole), polythiophene, poly(3-alkylthiophene), polyphenylene-vinylene, Polyaniline, poly(p-phenylene sulfide); carbon ink, carbon nanotube composites, and carbon nanotube nanofibers; metals and liquid metals. In particular, the protective coating and/or conductive coating—i.e. whichever coating is to contact the tissue—is chosen to have appropriate drag properties according to the proposed application.

Once the modulator or rectifying module and antenna is attached, as appropriate, the assembly of the modulator or rectifier and suture may be encapsulated in silicon, or relevant connections between the modulator or rectifier and antenna may be encapsulated.

The suture may therefore be very simply formed. Using these methods, conductive sutures may be fabricated with medical grade mechanical properties and biocompatibility. Due to their proximity to the surgical site, surgical sutures are a useful platform for integrating sensing capabilities into medical devices for monitoring the surgical site.

In a preferred embodiment, the suture is fabricated to have two conducting portions separated by an insulating portion, as shown in, for example, FIG. 5. In this embodiment, the conductive portion of the suture has the structure described above, whereas the insulating portion comprises the inner suture coated by the encapsulating material alone. This enables a single suture to be employed as both parts of the antenna 105 or both electrodes 5321, 5323, as appropriate.

Due to the potentially fatal consequences of failed medical equipment used during surgery, key to ensuring the surgical suture and other devices described herein are safe is their simplicity and inherent mechanical and functional properties—e.g. sutures described herein may, to the extent possible, maintain or mimic the inherent mechanical and functional properties of a medical grade suture.

As discussed above, in a particular embodiment, the fabrication of a WISE suture may involve a process by which a medical-grade surgical suture is coated with biocompatible conductive polymer, poly(3,4-ethylenedioxythiophene)-poly(styrenesulfonate) (PEDOT:PSS) and is then encapsulated with biocompatible polymer parylene-c. This process ensures the electrical conductivity of medical-grade sutures without compromising on the pliable mechanical properties and functionality of the suture. A highly miniaturized tag (either the modulator or modulator and antenna) with an RLC circuit can then be attached to the suture at a desired site before or after suturing. The sensing device including the modulator and antenna (i.e. suture), or the sensing device incorporating the antenna and modulator into the same component, may then be encapsulated with silicone.

FIG. 22 shows a schematic representation of a particular method of producing a conductive suture suitable for use as an antenna in accordance with an embodiment.

In step S1101 a medical grade suture is provided. In an embodiment the suture is silk. In other embodiments the suture may comprise other suitable materials such as cotton or vicryl, etc.

In step S1103 the suture undergoes an oxygen plasma treatment. For example, the suture may be placed inside an oxygen plasma chamber for at least two minutes. Oxygen plasma treatment has been found to advantageously improve the adsorption of the conductive coating onto the suture.

In step S1105 the suture is chemically treated to remove wax from the suture. In particular, the suture may be treated with N-Methyl-2-pyrrolidone (NMP). For example, the suture may be soaked in NMP for at least two minutes. Chemical treatment to remove wax has been found to advantageously improve the adsorption of the conductive coating onto the suture. Alternatively, DMSO could be employed for the chemical treatment.

Step S1107 the suture is coated with a conductive material. Suitable materials are listed above. In a particular embodiment, it is coated with PEDOT:PSS followed by drying under vacuum. In other embodiments the drying may be performed in an oven. Preferably the PEDOT:PSS is mixed with a dopant, such as 5% DMSO before coating.

In an embodiment step S1107 is performed between 1 and 5 times. In particular, it is performed at least 3 times. Three layers of coating has been found to advantageously reduce electrical resistance in the suture.

In step S1109, the suture is encapsulated with encapsulated with a biocompatible encapsulation material. In a particular embodiment, it is encapsulated with parylene-c, however other suitable encapsulating materials could be employed. Advantageously, paralyene-c ensures that the pliability of the inner suture is retained after encapsulation.

The above method has been found to advantageously produce conductive sutures suitable for simultaneous use as a suture and an antenna and/or electrode for devices according to embodiments described above.

It will be appreciated that one or more of the above steps may be omitted or additional steps may be added according to embodiments. It will also be appreciated that other conductive sutures having the required conductivity, strength and pliability properties could be employed with devices according to embodiments described above.

Experimental and Simulation Results

Particular non-limiting features of embodiments described above will now be illustrated using experimental and simulation results.

Simulations of a modulator according an embodiment configured to retransmit the second harmonic of a received signal at resonance frequency and sutures produced as described above in accordance with embodiments were performed in order to investigate the performance of WISE sutures. Three stitch patterns, Cushing stitch 2101, Lock-Stitch 2103 and Lembert stitch 2105 were employed.

Current distribution measurements indicated that current is distributed along the entire length of the stitches irrespective of the type of surgical stitch patterns at both fundamental and second harmonic frequencies.

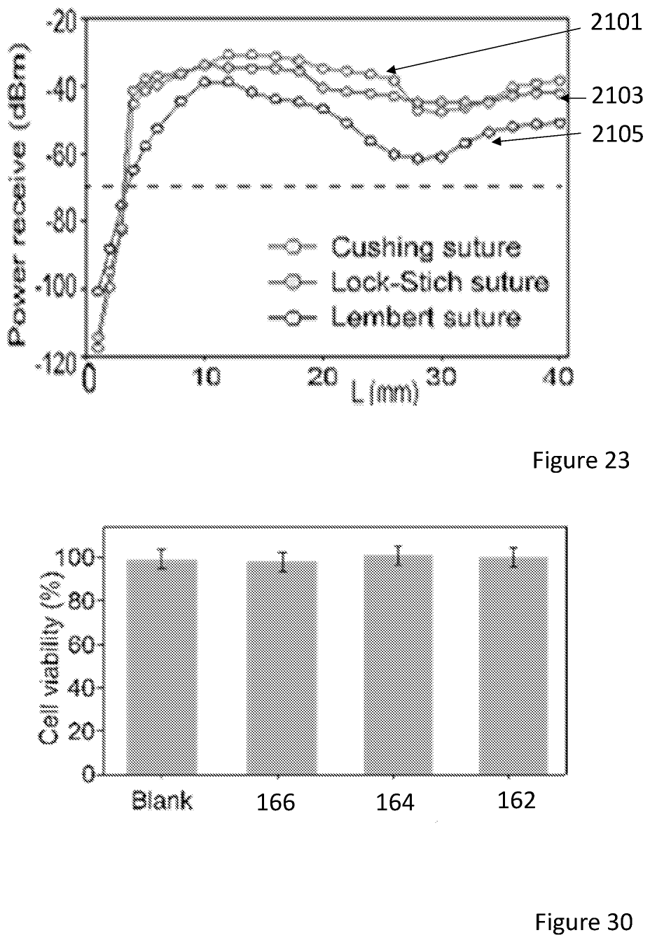

FIG. 23 shows receiving power of the suture as a function of the length of the suture for the three stitch patterns. The power received by the WISE sutures increases as the length of the stitch increases up to approximately 10 mm for all stitch patterns after which there is a slight drop off although the power remains largely constant thereon.

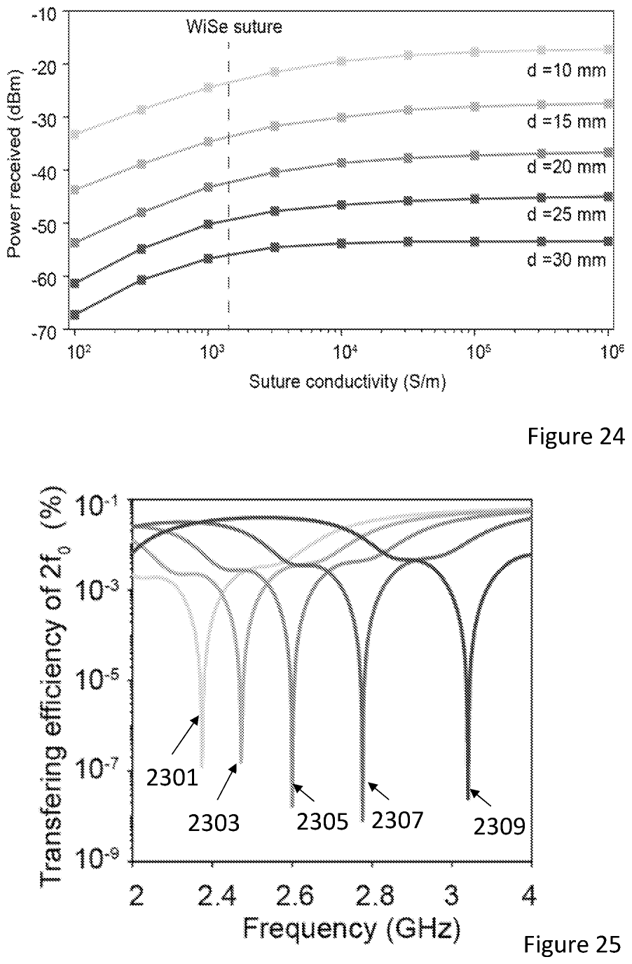

FIG. 24 shows the power received as a function of conductivity and diameter of the suture. It can be seen that power decreases with thickness.

FIG. 25 shows illustrates the transfer efficiency of WISE sutures as a function of frequency, and resonant frequency changes for capacitor capacities 2.7 pF (2301), 2.3 pF (2303), 1.9 pF (2305), 1.5 pF (2307) and 1.1 pF (2309).

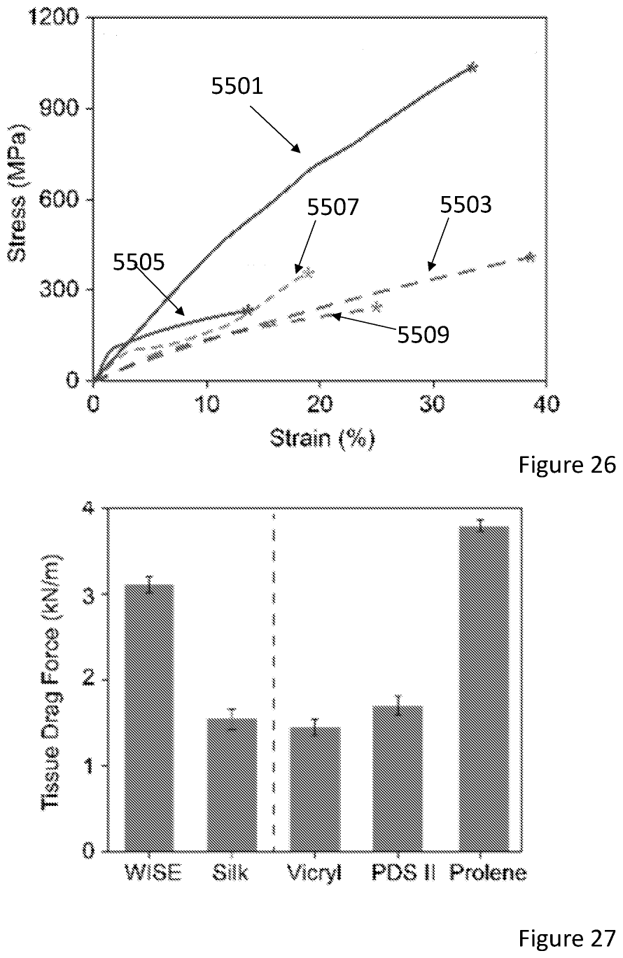

FIG. 26 shows the Stress (y-axis) as a function of strain (x-axis) for a number of commercially available sutures compared with a WISE suture produced in accordance with an embodiment. Results are shown for monofilament sutures Prolene 5501 and PDSII 5503, as well as braided silk 5505, Vicryl 5507 and WISE 5509 sutures The figure shows that the stress-strain plot of a WISE suture 5509 according to embodiments is close to that of a medical grade silk suture and within that of commercially available sutures. This is assisted, in particular, by starting from the foundation of a medical-grade suture and adding conductive functionality to it, as described above according to embodiments.

FIG. 27 shows the Tissue Drag Force for a number of commercially available sutures compared with a WISE suture produced in accordance with an embodiment. The figure shows that the tissue drag force exerted by a WISE suture according to embodiments is comparable to that of a medical grade commercial suture.

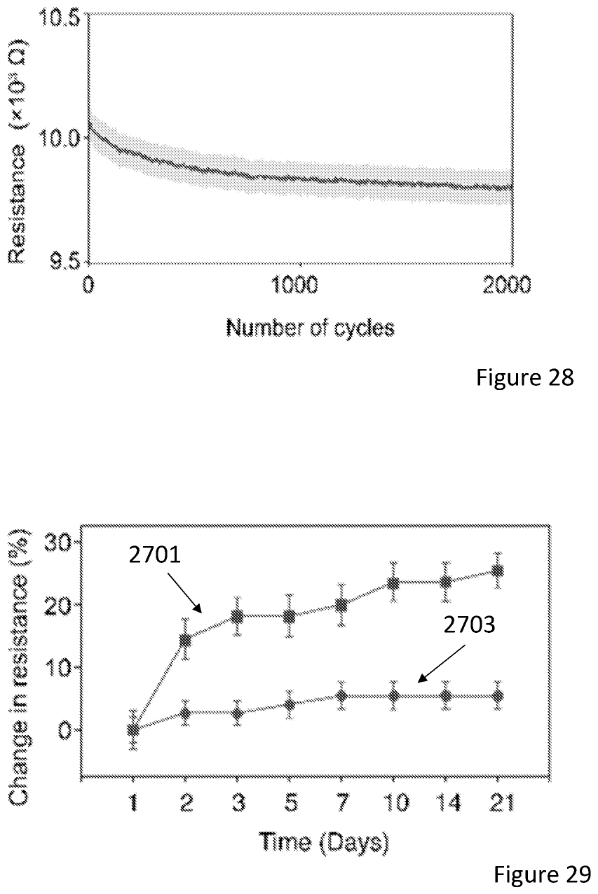

FIG. 28 shows the change in resistance of a WISE suture produced in accordance with an embodiment as the suture was subjected to mechanical cycles of contraction and elongation. As can be seen from FIG. 28, the suture was stable over 2000 mechanical cycles of contraction and elongation. In particular, the change in resistance was insignificant over 200 cycles, which means the electrical characteristics of the signal generator circuit should remain stable over a similar number of contraction and elongation cycles.

The change in resistance of WISE sutures produced in accordance with an embodiment was measured over three weeks in physiological buffer 1X phosphate buffer solution (PBS) and the results are shown in FIG. 29, with the top 2701 and bottom 2703 plots showing PEDOT:PSS coated sutures without and with parlyene-C encapsulation, respectively. The results show that WISE sutures were found to be stable over a period of 3 weeks with a % change in resistance less than 10%.

The biocompatibility of WISE sutures according to embodiments was compared to medical grade sutures. Human dermal fibroblasts (HDFs) were treated for 72 hours with a medical grade silk suture, PEDOT:PSS coated silk suture and a WISE suture produced in accordance with an embodiment. Confocal images with live HDFs showed that WISE sutures were not cytotoxic to HDFs.

FIG. 30 shows that the cell viability 162 for WISE sutures was 100% which equals or exceeds that for PEDOT:PSS coated 164 and silk 166, and is thus comparable with the biocompability of medical-grade sutures generally.

Sensing applications using WISE sutures to which modulators according to embodiments are attached were demonstrated in vivo and ex vivo. A WISE suture produced in accordance with embodiments was used to appose an incisional wound on the skin and deep inside the body of a mouse, with modulators attached either before or after suturing. Bacterial infection detection was integrated into the WISE sutures by application of a layer DNA-hydrogel produced according to the method of FIG. 12 to the modulator as described above in relation to FIG. 11.

The DNA hydrogel layered on the capacitor was found to is degraded by the extracellular nuclease secreted by Staphylococcus aureus bacteria within 10 hours following treatment with the bacteria like Staphylococcus aureus, the DNA hydrogel attached to the capacitor is degraded by the extracellular nuclease secreted by the bacteria within 10 hours.

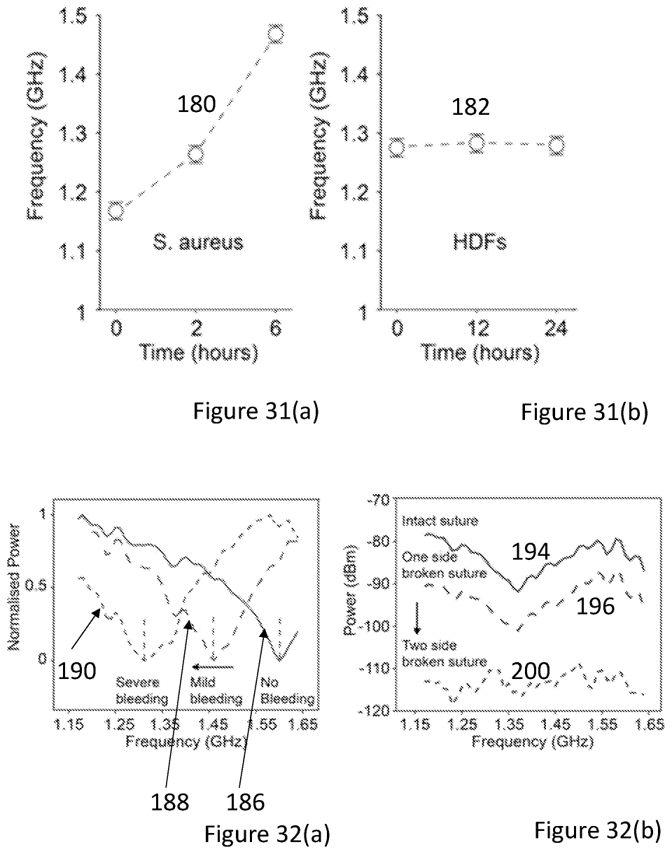

The Staphylococcus aureus progression produced a change in capacitance and in-turn a change in resonant frequency of the modulating circuit from 1.18 GHz to almost 1.5 GHz as shown in FIG. 31(a), reference 180, for Staphylococcus aureus.

A control experiment was conducted comprising treating the hydrogel with healthy human dermal fibroblasts (HDFs). The resonant frequency for this control group stayed stable around 1.28 GHz for almost 24 hours as shown in FIG. 31(b) reference 182, in contrast to the change seen in FIG. 31(a).

The change in resonant frequency of a modulating circuit according to an embodiment was explored under conditions of bleeding. The results are shown in FIG. 32(a) which shows the Normalised Power as a function of Frequency for three different severities of bleeding using a WISE suture produced according to embodiments in combination with a modulator.

When there is sudden haemorrhage—190—the permittivity of the area over the capacitive part of the modulating circuit changes, causing shift in resonant frequency. The shift from the unaffected condition 186 becomes more obvious as the sensor gets completely saturated, as the frequency shifts from 1.6 GHz to the affected condition at 1.45 GHz for mild bleeding—188—and to the affected condition at 1.3 GHz for severe bleeding—190. In this embodiment, the affected condition therefore constitutes multiple conditions that are not the unaffected condition, or is indicative of a range of an event (e.g. mild to severe bleeding) the whole of which represents an affected condition.

The effect of a suture breakage was also explored. When a WISE suture produced in accordance with embodiments in combination with a modulator according to an embodiment was used to appose a surgical site, a break in the suture of FIG. 32(b)—resulted in the power of the device decreasing from the intact power 194, as observed as a drop from −85 dBm to −95 dBm (reference 196) for a break on a single side of the modulator (i.e. the suture 198 extends from both sides of the modulator and a break appears on one side of the modulator only) and to −115 dBm (reference 200) for a break across two sides of the suture. No power was detected as the suture completely broke. The resonant frequency remained constant as long as the sensor was intact. The decrease in power is due to a compromise in suture integrity and shows that the conductivity of WISE sutures can also be used to sense change of events. This communicates to or alerts the clinician, patient and/or care-taker that the suture integrity has been compromised.

In vivo studies were done as per IACUC standards using Sprague Dawley (SD) male rats to demonstrate the wound healing capability and device stability of WISE sutures produced in accordance with embodiments on the skin and in the muscle. The rats were euthanized on days 1, 4, 7 and 14 post-surgery to study the histopathological events of wound healing process. The histopathological staining by Haemotoxylin and Eosin (H&E) staining process revealed that WISE sutures were similar to medical grade sutures as they elicited the exact histological events that occur during a normal, healthy acute wound healing process for 14 days. Observations taken on day 1 showed necrosis and inflammatory cells around the incisional wound site, day 4 and 7 showed granulation tissue formation and wound healing and day 14 showed complete re-epithelialization and wound closure.

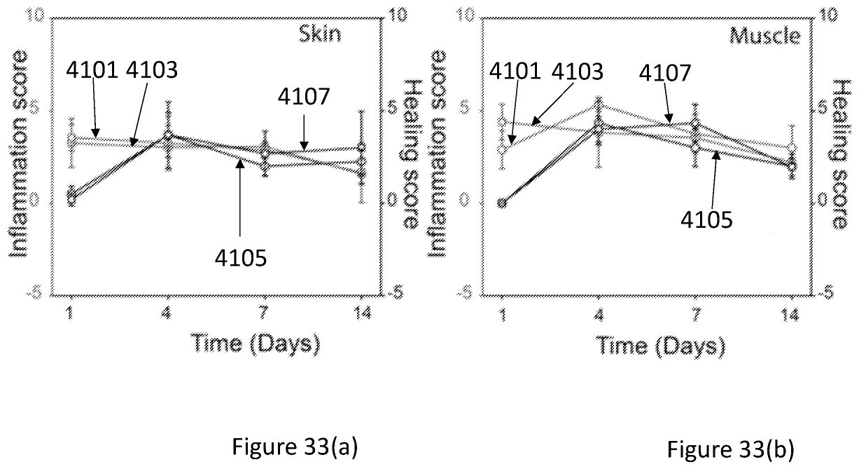

FIGS. 33(a) and 33(b) show the obtained inflammation (left axis) and healing scores (right axis), for skin and muscle, respectively, over 14 days, each figure showing the results for an inflammation control 4101, an inflammation test 4103, a healing control 4105 and an inflammation test 4107. The inflammation and healing increased from day 1 to day 4 and decreased towards day 14 both for skin and muscle.

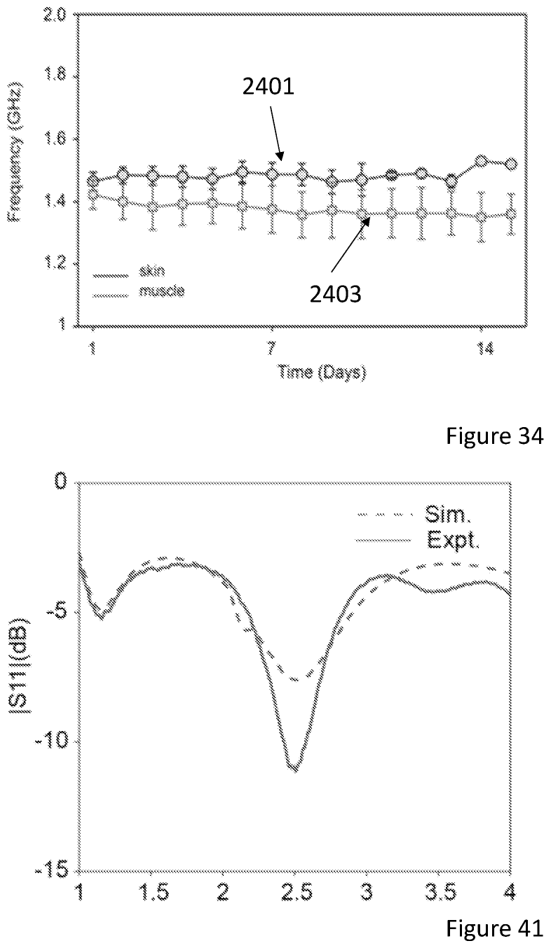

The resonance frequency of the device was also measured over the 14 days and the results are shown in FIG. 34 (2401 indicating skin and 2403 indicating results for muscle). The results show that the resonance frequency was stable for the entire wound healing phase of 14 days. This demonstrates the stability of WISE sutures comprising conductive sutures and an attached modulator.

The optimization of WISE suture preparation was explored using 5 different protocols shown in Table 1.

TABLE 1

| Protocol | | | |
|---|---|---|---|
| No. | | Absorption | Resistance |
| 1 | PEDOT:PSS | Few | Very high |
| | coating with oven | PEDOT | resistance |
| | drying (4 times) | absorption | of ~10 MΩ/cm |
| 2 | Dopant (5% | Few | High |
| | DMSO) + | PEDOT | Resistance |
| | Protocol 1 | absorption | of ~100 KΩ/cm |
| 3 | Chemical (NMP) | Moderate | Lowered |
| | treatment to | PEDOT | resistance |
| | remove wax + | absorption | of ~10 KΩ/cm |
| | Protocol 2 | | |
| 4 | Oxygen Plasma | Good | Even lower |
| | treatment + | PEDOT | resistance |
| | Protocol 3 | absorption | of ~1 KΩ/cm |
| 5 | WISE suture | Best | Lowest |
| | protocol:Protocol | PEDOT | resistance |
| | 4 + vacuum drying | absorption | of ~100 Ω/cm |

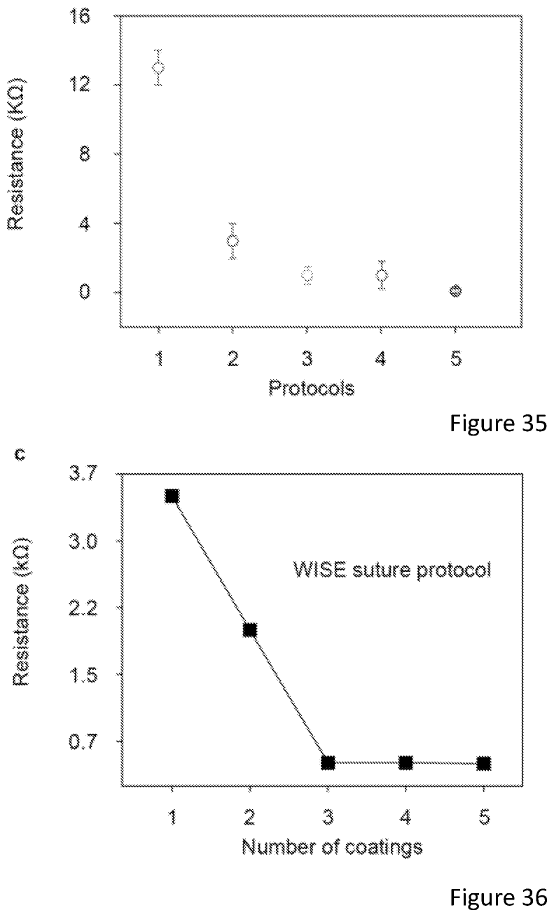

The resistance of the sutures prepared with each protocol was investigated and shown in FIG. 35, with error bars indicating the standard deviation (three sutures were prepared with each protocol).

FIG. 36 shows the resistance of five sutures prepared with protocol 5 of Table 1 but with different numbers of coatings of PEDOT:PSS applied, as indicated on the x-axis. The resistance remained approximately stable above three coatings.

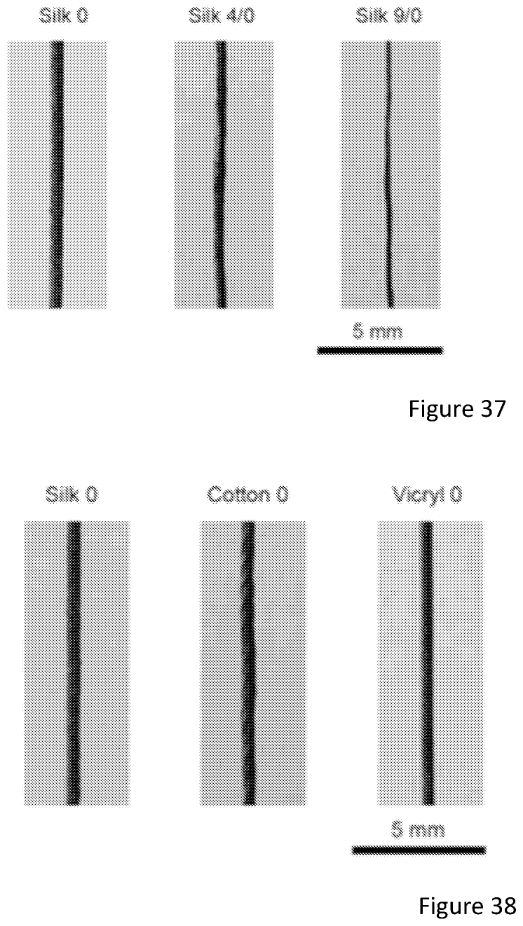

PEDOT:PSS coated silk sutures of three different sizes were successfully prepared using the protocol 5 of Table 1, as shown in the images of FIG. 37. Likewise, three sutures of the same size (0) were produced using the protocol 5 of Table 1 but with different base sutures of silk, cotton and vicryl, as shown in FIG. 38. Thus, the methods of producing conductive sutures according to embodiments are suitable for a range of suture sizes and can be extended to other medical grade sutures beyond silk. Note that the Suture size indicated follows the designation by United States Pharmacopeia (U.S.P.).

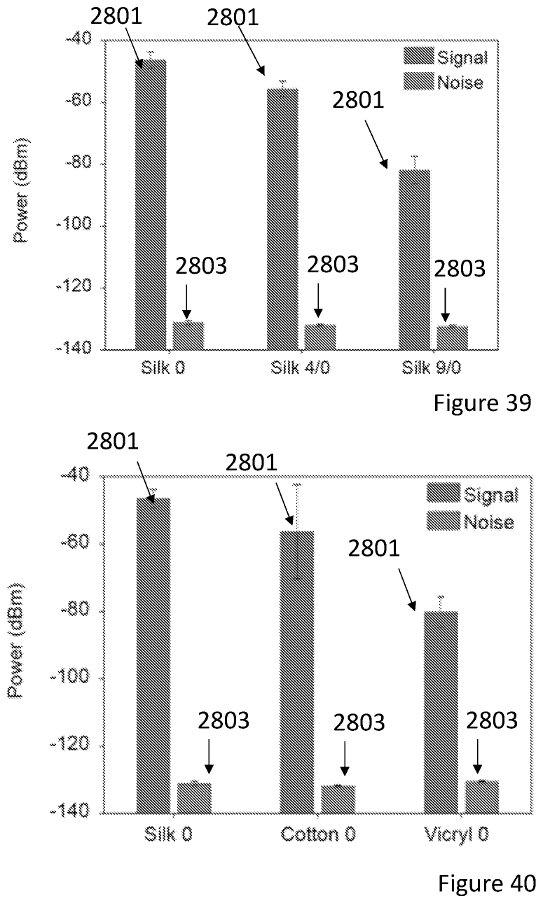

The harmonic signal of the prepared sutures was measured and the resulting signal 2801 and noise 2803 power measurements are shown in FIGS. 39 and 40. The results show that all sutures give a large signal to noise ratio, with best results obtained for size 0 silk sutures.

The performance of the reader antenna according to the embodiment of FIGS. 19(a) and 19(b) was simulated and tested experimentally. The results of shown in FIG. 41 as the wireless reflection coefficient S11 as a function of frequency. The results show good performance on all frequencies explored, including lower frequencies.

The maximum detection depth of WISE sutures with a suture produced in accordance with embodiments in combination with a modulator was investigated for three stitch types: Lembert, Lock-stitch and Cushing. For the Lembert suture, the maximum detection depth for 10 dB SNR was found to be approximately 5 cm, whereas for the lock-stitch and Cushing sutures it was found to be approximately 6 cm at the optimal length L (i.e. where the WISE suture works as a resonant dipole antenna with maximum power transmission efficiency). It is notable that the optimal detection depth can be achieved by selective functionalization of suture or be tuned by operation at different frequency, in favour of monitoring deep surgical sites. Moreover, the suture length dependence of wireless signal seen supported the proposed interrogation of suture breakage via wireless method according to embodiments.

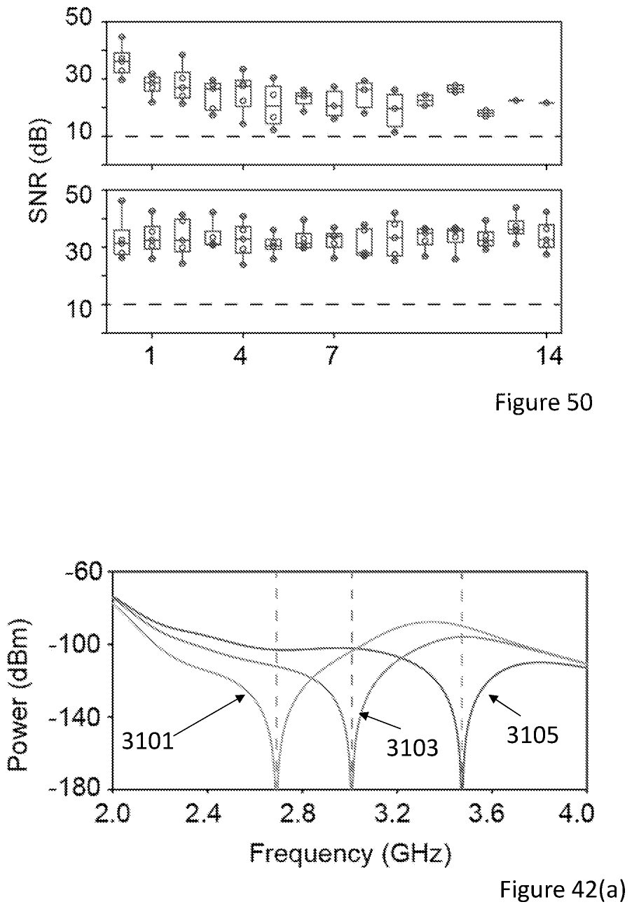

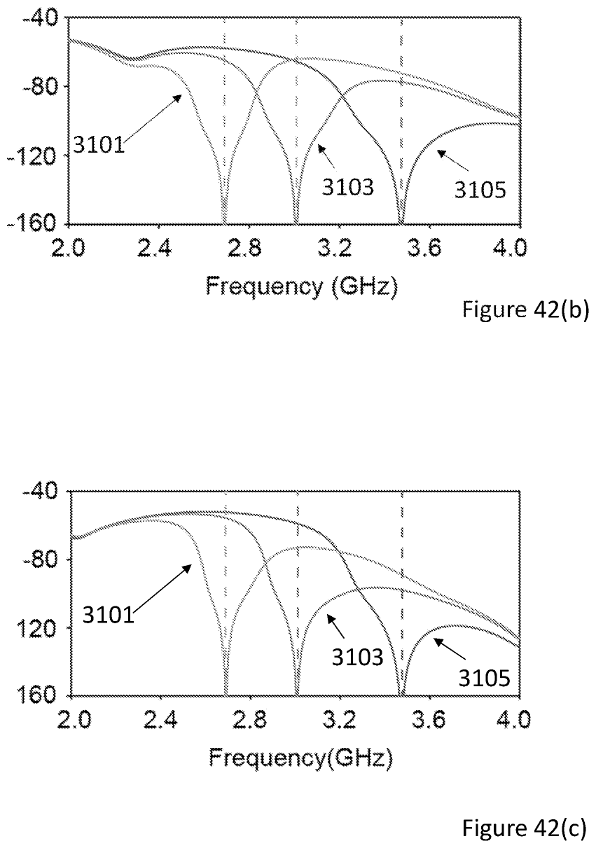

FIGS. 42(a), 42(b) and 42(c) show simulated harmonic spectra for the three different sutures, respectively with length L=20 mm and d=25 mm. The capacitance of the integrated modulating circuit was computationally adjusted to three different values 1.0 pF (3101), 0.8 pF (3103) and 0.6 pF (3105) to mimic the varied sensor states. The obtained harmonic spectra demonstrate clear shift (>0.32 GHz) of resonant frequency with 0.2 pF change in sensor capacitance, supporting the proposed frequency sensing mode according to embodiment. The simulation results demonstrate the tunability of the WISE platform, offering a guidance for WISE suture design and optimization.

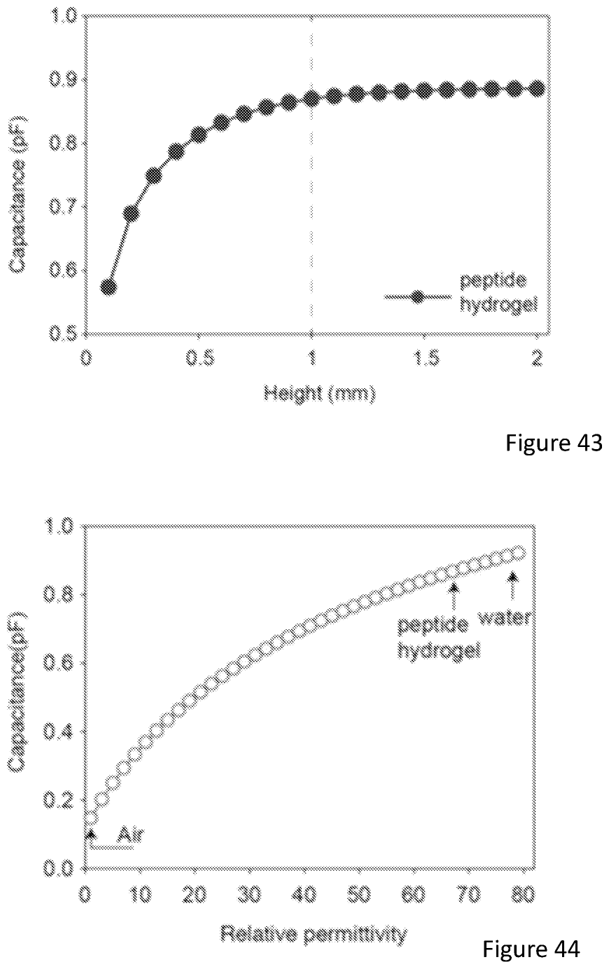

A simulation model according to the schematic of FIG. 11 was produced to simulate the modulating circuit response with varying hydrogel thickness. The simulation model was based on a 25 μm polyimide substrate, 18 μm copper electrodes (Pyralux® AC) and 0.1 mm medical grade silicone (Kwil-Sil, WPI) as the surface encapsulation. Two PDMS pillars provided necessary mechanical support for hydrogel in vivo. FIG. 43 shows the simulated capacitance of the modulating circuit loaded with a cylinder shape of peptide hydrogel with d=2 mm, showing that the capacitance decreases as the height of the hydrogel decreases.

FIG. 44 shows the simulated capacitance of the modulating circuit in contact with cylindrical shape media (d=2 mm, h=1 mm). These results demonstrate that the capacitance variation and sensitivity can be tuned by changing the gap between interdigital electrodes and the thickness of silicone coating, thereby altering the relative permittivity.

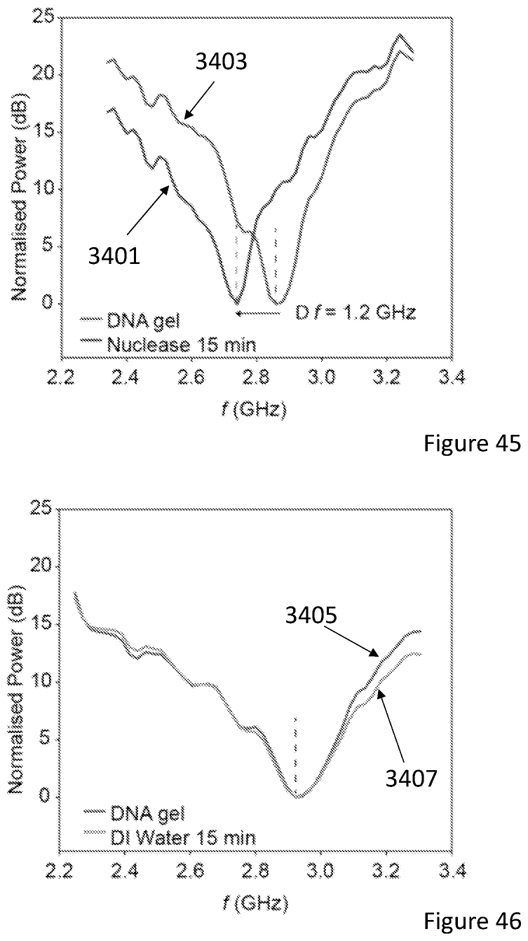

FIG. 45 shows the effect of the addition of nuclease on the resonant frequency of the simulated modulating circuit described above with a layer of DNA hydrogel produced in accordance with the method of FIG. 12 on the capacitor. The spectrum is shown before (3403) and after (3401) the addition of 10 μL nuclease (1000 units/mL) degraded the DNA hydrogel on the electronic pledget within 15 minutes, causing a shift of resonant frequency ˜1.2 GHz.

In comparison, FIG. 46 shows the effect of the addition of 10 μL DI water on the resonant frequency of the simulated modulating circuit with DNA gel described above. The spectrum is shown before (3405) and 15 minutes after (3407) the addition of 10 μL DI water. No detectable effect is observed, i.e. no shift in resonant frequency.

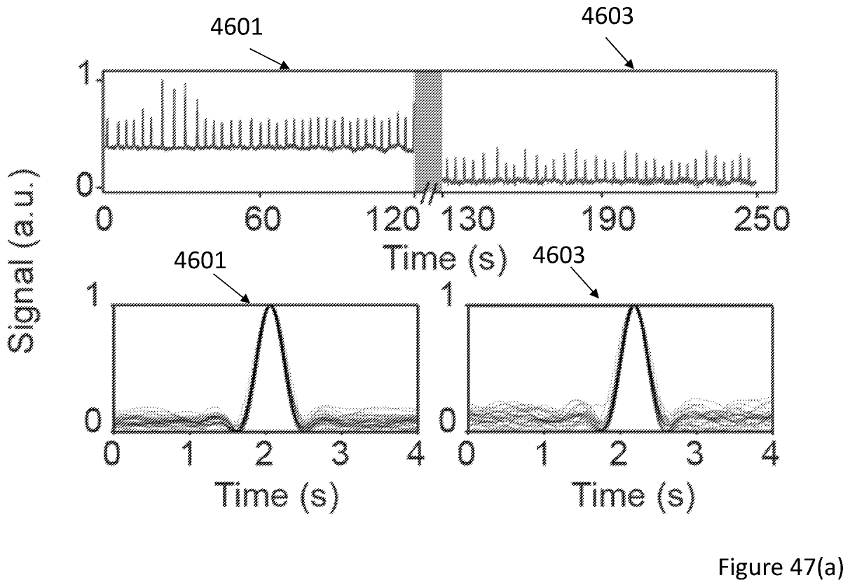

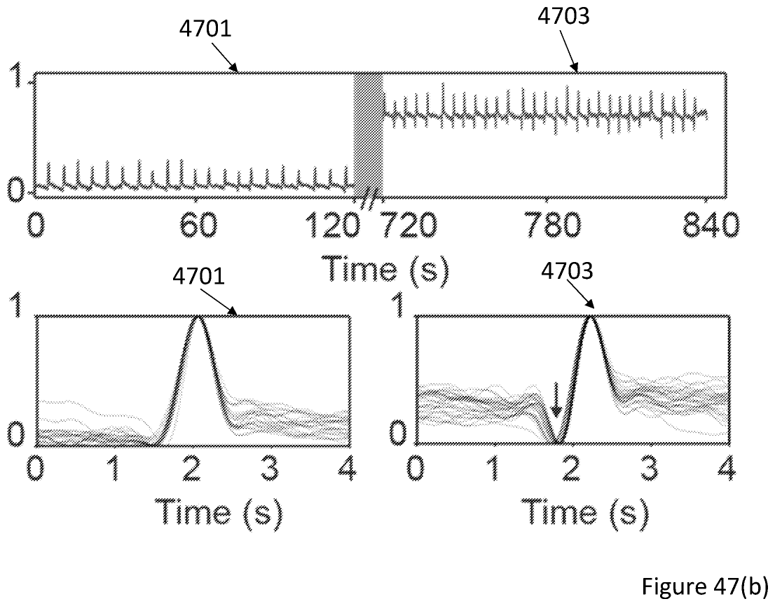

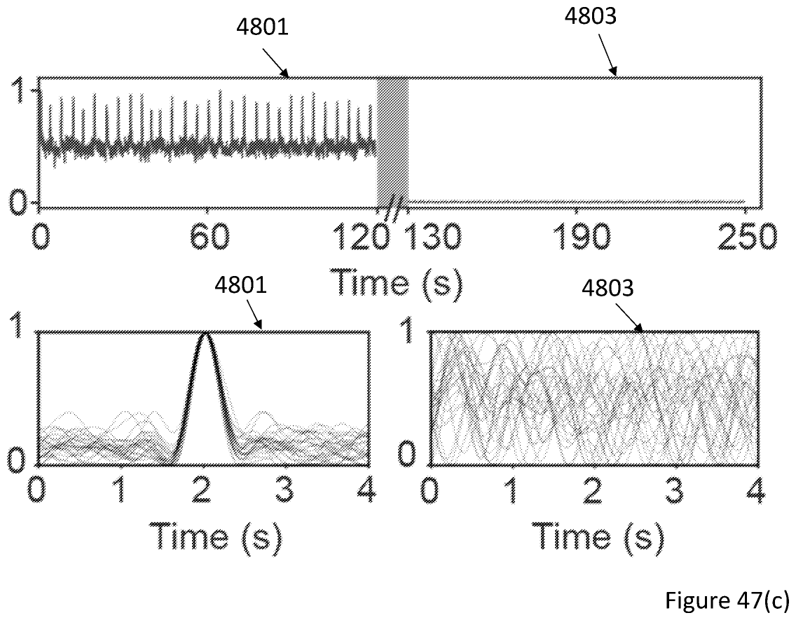

FIGS. 47(a), 47(b) and 47(c) demonstrate dynamic vital sign monitoring using a simulated WISE suture according to an embodiment. The relative distance d between transmitter and receiver is encoded by the vibratory motion of physiological processes, thus the vital signs, such as respiratory rate, can be captured by the change of WISE signal amplitude. The figures show the signal received before 4601 and after 4603 (FIG. 47(a)) skin closure, (FIG. 47(b)) before 4701 and after 4703 gastric solution injection and (FIG. 47(c)) before 4801 and after 4803 suture breakage (top panels). The lower panels show the signals after normalisation for clarity.

Continuous wavelet transform (CWT) spectrograms were also obtained, enabling the extraction of the respiratory rate (RR) of a rat.

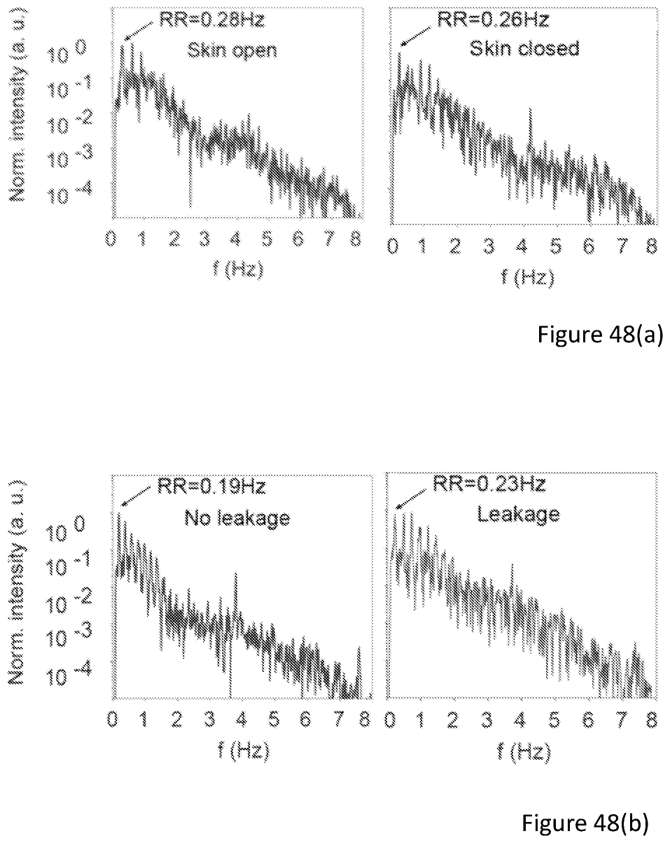

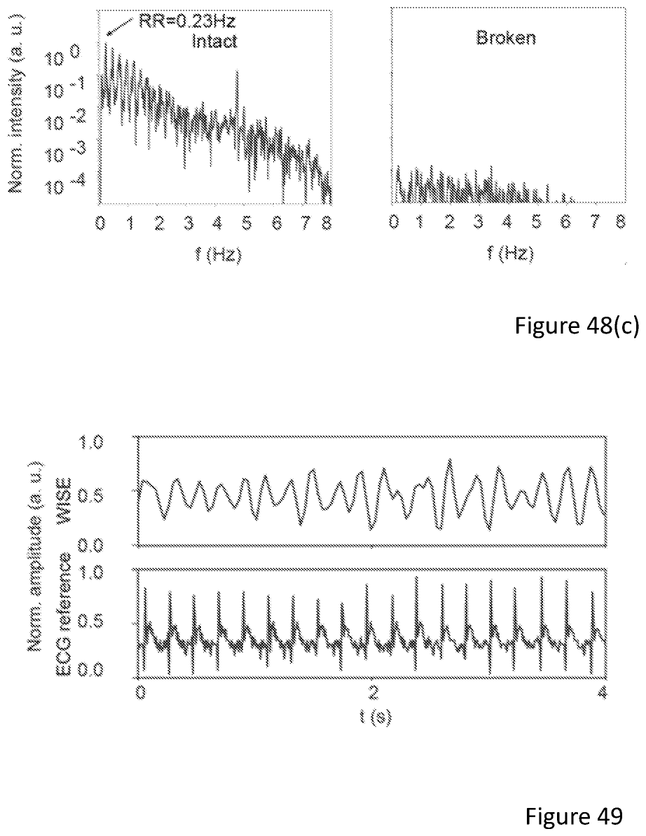

FIGS. 48(a), 48(b) and 48(c) show the frequency spectra obtained from WISE sutures according to embodiments and show spectra before and after skin closure (FIG. 48(a)), before and after exposure to gastric solution (FIG. 48(b)) and before and after suture breakage (FIG. 48(c)) showing that vital sign measurements are possible in all cases except suture breakage.

FIG. 49 shows a comparison of the amplitude of a backscatter signal measurement taken using a WISE suture (top panel) compared with an ECG signal.

Harmonic spectra were obtained experimentally with a WISE suture according to an embodiment on skin and muscle respectively over 14 days. In both cases, the obtained spectrum demonstrated good stability. FIG. 50 shows the signal to noise variation over the same period for skin (top panel) and muscle (bottom panel) obtained from 5 samples (error bars indicate standard deviation). Note that for the skin group, only one device was left at 13 and 14 days due to rat scratching. Again, the SNR remains relatively consistent over the period, particularly for muscle.

As shown, to achieve real-time monitoring of, for example, a surgical site, wireless sensing (WISE) surgical sutures have been developed that can monitor the surgical site for surgical wound dehiscence and subsequent post-surgical complications like compromise in suture integrity, sudden haemorrhage/bleeding and bacterial infection and also simultaneously monitor the wound healing status and communicate the same. Some sutures disclosed herein overcome challenges through two key advances: (i) functionalizing medical grade sutures by coating with biocompatible conductive polymer, poly(3,4-ethylenedioxythiophene)-poly(styrenesulfonate) (PEDOT:PSS) which renders the sutures electrically conductive without compromising on pliable mechanical properties and functionality, and (ii) the sutures wirelessly transmit information to an external device through a harmonic backscatter technique in which highly miniaturized electronics (less than 1 mm² in the smallest version), comprising of RLC based sensor, modulate the signal reflected by the conductive suture.

Since the wireless sensing (WISE) technology non-invasively communicates information about the events occurring at remote regions to an external device through harmonic backscattering, wires can be eliminated. This involves a highly miniaturized transponder. Moreover, the tests described above show this concept is realisable, using sutures fabricated with medical grade mechanical properties and biocompatibility. The proof of concept experiments demonstrate the capability of the WISE sutures to sense bleeding, infection and compromise in suture integrity in real time. The RLC based sensors used as the modulating circuit of the modulators attached to the WISE sutures can sense and monitor any surgical complication real-time. WISE sutures are also capable of stimulating nerves, eluting drugs and performing other similar theranostic applications. The in vivo studies show that the wound healing process is not deterred by WISE sutures and are similar to medical grade sutures in eliciting the histopathological events of wound healing. WISE sutures are stable in wireless performance throughout the period of 14 days inside the animal body.

The present technology may be part of, incorporated into or added to, a medical device such as a bandage, stent, valve, prosthesis or other medical implant or device. For example, a conductive thread can be incorporated into a bandage and a transmission device can then be attached to the conductive thread the same way as for the suture embodiment. In another example, a transponder may be attached to a stent which itself will generally be formed from conductive material. The technology may also be incorporated into food packaging—e.g. attached to an internal surface of the packaging—to monitor for growth of bacteria that regularly grow in packaged foods.

As described above, the present transmission device with, or attached to, a radiofrequency suture can be used to monitor the remote site on-demand and continuously for changes in environment. One of the applications of the invention is to monitor the surgical site for post-surgical complications like bleeding, infection, compromise in suture integrity etc. It can also be used to monitor food degradation, for example, by being incorporated into packaging, etc. The device may be configured such that its capacitance or other electrical properties of the device change in the event of food spoilage and a handheld reader may be employed to power and communicate with the device. For example, the device may be coated or a portion of it may be coated with a hydrogel which is susceptible to degradation in the presence foodborne bacteria in an analogous fashion to the in-vivo applications using a hydrogel described above in association with FIGS. 11 to 16.

Other uses of devices according to embodiments include veterinary surgical site monitoring, crop physiology and agricultural monitoring such as soil monitoring.

Advantageously, where post-surgical complications are usually realized very late and call for invasive and expensive corrective methods, embodiments of the present invention may eliminate the need for such measures, as the complications can be wirelessly sensed real-time and thus identified early. Moreover, the efficiency of the present device is sufficient to safely power the device inside the body.

The use of harmonic backscattering in transponder embodiments means the signal received by the remote device (e.g. portable, hand-held device) is readily distinguishable from the signal emitted by that device. By using passive charging, very small batteries or energy harvesting devices, sensors can be powered with little or no battery power.

It will be appreciated that many further modifications and permutations of various aspects of the described embodiments are possible. Accordingly, the described aspects are intended to embrace all such alterations, modifications, and variations that fall within the spirit and scope of the appended claims.

Additional embodiments of the invention are given in the following statements:
1. A transmission device comprising:
   an antenna connector for connecting to an antenna; and
   a signal generator for generating a signal for transmission using the antenna when the transmission device is attached thereto, wherein the signal generator has an unaffected condition and an affected condition, and predetermined conditions around the transmission device cause the signal generator to transition to the affected condition, the signal when generated by the signal generator in the unaffected condition being different to the signal if generated by the signal generator when in the affected condition.
2. The transmission device of 1, being a transponder.
3. The transmission device of 1 or 2, wherein the predetermined conditions comprise the growth of bacteria.
4. The transmission device of 3, wherein the signal generator comprises a coating that is susceptible to consumption by the bacteria.
5. The transmission device of 4, wherein the coating comprises a bacterium-specific deoxyribonucleic acid (DNA) hydrogel.
6. The transmission device of 1 or 2, wherein the predetermined conditions comprise blood leakage onto the transmission device.
7. The transmission device of any one of 1 to 6, wherein the predetermined conditions comprise a compromise of the antenna.
8. The transmission device of 7, wherein the predetermined conditions comprise a break of the antenna and, on break of the antenna, the signal generator fails to transmit the signal.
9. The transmission device of any one of 1 to 8, wherein the signal is generated by harmonic backscattering.
10. The transmission device of 1 or 2, wherein the predetermined conditions comprise dehiscence.
11. The transmission device of any one of 1 to 10, wherein the antenna comprises a conductive suture.
12. The transmission device of 4 or 5, being adapted for placement in food packaging, wherein the coating is selected for consumption by a food-borne bacterium.
13. The transmission device of any one of 1 to 12, further comprising the antenna, wherein the signal generator is activated by a remote device, the remote device activating the signal generator by transmitting a signal of a resonant wavelength of the signal generator, that is captured by the antenna.
14. The transmission device of any one of 1 to 12, wherein the signal generator is activated by electromagnetic field applied by a remote device.
15. The transmission device of any one of 1 to 14, wherein the signal generator is an inductor-capacitor circuit, the inductance and/or capacitance changing as the signal generator transitions to the affected condition.
16. The transmission device of any one of 1 to 15, comprising the antenna, the antenna connector connecting the signal generator about a centre of a length of the antenna, the transmission device being adapted to be positioned in a surgical site, wherein the signal generator transitions to the affected condition during healing at the surgical site.
17. A transmission assembly comprising:
   a transmission device according to any one of 1 to 16; and
   the antenna connected to the signal generator by the antenna connector.
18. An electrically conductive suture, comprising a surgical suture apposing tissue portions, the suture being coated in a conductive coating, the coated surgical suture being encapsulated in a protective coating.
19. The suture of 18, wherein the protective coating is an inert coating.
20. The suture of 18 or 19, wherein the protective coating is biocompatible polymer.
21. The suture of 20, wherein the biocompatible polymer is parylene c.
22. The suture of any one of 18 to 21, wherein the conductive coating is a biocompatible conductive polymer.
23. The suture of 22, wherein the biocompatible conductive polymer is poly(3,4-ethylenedioxythiophene)-poly(styrenesulfonate).
24. A method for forming an electrically conductive suture, comprising:
   providing a medical-grade suture;
   coating the medical-grade suture in a conductive coating; and
   coating the coated, medical-grade suture in a protective, non-conductive coating.
25. A medical device comprising a transmission device according to any one of 1 to 16, or a transmission assembly according to 17.
26. The medical device of 25, being one of a suture, bandage, stent, valve, and prosthesis.

1. A wirelessly triggered device for implantation in vivo comprising:
an electrically conductive suture;
an electronic circuit coated with a biocompatible encapsulating material and communicatively coupled to the electrically conductive suture, the electronic circuit arranged to convert a received wireless triggering signal into an electrical signal for passing through the conductive suture.

2. (canceled)

3. (canceled)

4. (canceled)

5. (canceled)

6. (canceled)

7. (canceled)

8. The wirelessly triggered device according to claim 1, wherein the electrically conductive suture is arranged to receive the wireless triggering signal, and the electronic circuit includes a modulating circuit operable to modulate the received wireless triggering signal to produce a backscatter response signal having a specific harmonic as the electrical signal, for transmission by the electrically conductive suture.

9. The wirelessly triggered device according to claim 8, wherein the wirelessly triggered device is a sensing device for monitoring conditions at a site, further comprising a detector operable to detect a predetermined condition at the site, and wherein the modulating circuit is operable to modulate the backscatter response signal based on the detected predetermined condition.

10. The wirelessly triggered device according to claim 9, wherein the detector includes a passive component of the modulating circuit.

11. The wirelessly triggered device according to claim 1, wherein the electronic circuit includes a rectifier operable to rectify the wireless triggering signal to produce an electrical current as the electrical signal, the electrical current being passed through the conductive suture; and
an antenna for receiving the wireless triggering signal, the rectifier being communicatively coupled to the antenna, wherein the antenna comprises the electrically conductive suture or a further electrically conductive suture.

12. (canceled)

13. (canceled)

14. (canceled)

15. (canceled)

16. A wirelessly triggered device for monitoring conditions at a site, comprising:
a detector operable to detect a predetermined condition at the site;
a modulating circuit configured to be communicatively coupled to an antenna, the modulating circuit operable to modulate a wireless triggering signal received at the antenna to produce a backscatter response signal having a specific harmonic, for transmission by the antenna, based on the detected predetermined condition,
wherein the detector includes a passive component of the modulating circuit.

17. The wirelessly triggered device according to claim 16, where the wirelessly triggered device is a passive electronic device.

18. The wirelessly triggered device according to claim 16, further comprising the antenna and wherein the wirelessly triggered device comprises a printed circuit board and wherein the modulating circuit and the antenna are printed onto the printed circuit board.

19. (canceled)

20. (canceled)

21. (canceled)

22. The wirelessly triggered device according to claim 16, further comprising a connector for connecting the wirelessly triggered device to a wound closure device.

23. (canceled)

24. (canceled)

25. (canceled)

26. (canceled)

27. The wirelessly triggered device according to claim 10, wherein the predetermined condition comprises one or more adverse physiopathological states.

28. The wirelessly triggered device according to claim 27 wherein the one or more adverse physiopathological states includes one or more of bleeding, bacterial infection, gastric juice leakage and anastomotic leakage.

29. The wirelessly triggered device according to claim 10, wherein the modulating circuit comprises an RLC circuit and a resonance frequency of the RLC circuit varies based on the detected predetermined condition.

30. (canceled)

31. The wirelessly triggered device according to claim 10, further comprising a support member for supporting a layer of responsive material which is susceptible to undergo a change in the predetermined condition, the support member configured to support the responsive material over the passive component.

32. (canceled)

33. (canceled)

34. The wirelessly triggered device according to claim 31, wherein the responsive material comprises a hydrogel.

35. The wirelessly triggered device according to claim 34, wherein the predetermined condition includes bacterial infection and the responsive material comprises a DNA hydrogel susceptible to degradation in the presence of nuclease secreted by bacteria.

36. The wirelessly triggered device according to claim 34, wherein the predetermined condition includes gastric juice leakage and the responsive material comprises a peptide hydrogel susceptible to degradation in the presence of pepsin.

37. (canceled)

38. (canceled)

39. (canceled)

40. (canceled)

41. (canceled)

42. (canceled)

43. (canceled)

44. (canceled)

45. (canceled)

46. (canceled)

47. (canceled)

48. (canceled)

49. (canceled)

50. (canceled)

51. (canceled)

52. A method of monitoring conditions at a site in vivo,
the method comprising:
implanting the wirelessly triggered device according to claim 8 into the site;
transmitting a plurality of interrogation signals configured to stimulate the backscatter response signal from the wirelessly triggered device;
receiving the backscatter response signal from the wirelessly triggered device; and
determining a condition at the site based on the backscatter response signal.

53. The method according claim 52, wherein the condition comprises one or more physiopathological conditions.

54. The method according to claim 52, wherein the condition includes one or more of healing, bleeding, infection, dehiscence, suture breakage, heart rate and respiration rate.

55. The method according to claim 52, wherein implanting the wirelessly triggered device comprises suturing at least a portion of a wound with the electrically conductive suture.

56. (canceled)

57. (canceled)
